(19) United States
(12) Patent Application Publication
(10) Pub. No.: US 2024/0198017 A1
(43) Pub. Date: Jun. 20, 2024

(54) VAPORIZER DEVICE WITH HYBRID HEATER

(71) Applicant: Pax Labs, Inc., San Francisco, CA (US)

(72) Inventors: Eric Brown, San Francisco, CA (US); David Carlberg, Portland, OR (US); Scott Collins, San Francisco, CA (US); Alexander Ringrose, Alamo, CA (US); Matthew J. Taschner, Bend, OR (US); Brian M. Warren, San Francisco, CA (US); Alexander Weiss, Oakland, CA (US)

(21) Appl. No.: 18/391,519

(22) Filed: Dec. 20, 2023

Related U.S. Application Data
(60) Provisional application No. 63/517,806, filed on Aug. 4, 2023.
(60) Provisional application No. 63/476,376, filed on Dec. 20, 2022.

(51) Int. Cl. A61M 11/04

(52) U.S. Cl. CPC A61M 11/042

(57) ABSTRACT

Features relating to a vaporizer device including a reusable vaporizer device body are provided. The vaporizer device includes a hybrid heating system including a convection heater and/or a conduction heater for rapidly heating vaporizable material stored in the vaporizer device.

### RELATED APPLICATION

This application claims priority to U.S. Provisional Application 63/476,376, entitled “VAPORIZER DEVICE WITH HYBRID HEATER,” and filed on Dec. 20, 2022, and U.S. Provisional Application 63/517,806, entitled “VAPORIZER DEVICE WITH HYBRID HEATER,” and filed Aug. 4, 2023, the disclosures of which is incorporated herein by reference in their entirety.

### TECHNICAL FIELD

The current subject matter described herein relates generally to vaporizer devices, such as portable, personal vaporizer devices for generating and delivering an inhalable aerosol from one or more vaporizable materials.

### BACKGROUND

Vaporizing devices, including electronic vaporizers or e-vaporizer devices, allow the delivery of vapor and aerosol containing one or more active ingredients by inhalation of the vapor and aerosol. Electronic vaporizer devices are gaining increasing popularity both for prescriptive medical use, in delivering medicaments, and for consumption of nicotine, tobacco, other liquid-based substances, and other plant-based smokeable materials, such as cannabis, including solid (e.g., loose-leaf or flower) materials, solid/liquid (e.g., suspensions, liquid-coated) materials, wax extracts, and prefilled pods (cartridges, wrapped containers, etc.) of such materials. Electronic vaporizer devices in particular may be portable, self-contained, and convenient for use.

### SUMMARY

Aspects of the current disclosure relate to vaporizer devices that include various features for improving aerosol production by the vaporizer devices and for improving the user experience while using the vaporizer devices.

In some embodiments, there is provided a vaporizer device including a mouthpiece; a hybrid heating system comprising a convection heater and a conduction heater; an oven chamber configured to contain a vaporizable material, wherein the vaporizable material is configured to be heated by convection, via air heated by the convection heater and by conduction, via contact with the oven chamber, wherein the oven chamber is configured to be heated by conduction using a heating element of the conduction heater, the heating element being in contact with the oven chamber; a vaporizer body comprising an outer shell which surrounds at least in part one or more internal components of the vaporizer body, the vaporizer body including a first end at which the mouthpiece is positioned and a second end opposite the first end, wherein the outer shell extends between the first end and the second end, the outer shell having two opposing long side portions and two opposing short side portions that define the outer shell, wherein the outer shell includes a recess exposing an interior of the vaporizer body, wherein the oven chamber is positioned in the vaporizer body and is exposed by the recess; and a vaporizer cover configured to be removable from the vaporizer body and configured to enclose at least the recess and the oven chamber.

In some variations of the methods, systems, and non-transitory computer readable media, one or more of the following features can optionally be included in any feasible combination. In some variations, the recess is positioned along a length of the vaporizer body. The length runs parallel to the two opposing long side portions of the vaporizer body. Long sides of the vaporizer cover are parallel to the two opposing long side portions of the outer shell. The vaporizer cover includes an outer surface and an inner surface, wherein the outer surface is configured to face away from the vaporizer body, wherein the inner surface is opposite the outer surface and is configured to face towards an interior of the vaporizer body. The inner surface includes a solid projection. The solid projection extends from the inner surface into an interior volume of the oven chamber to enable compression of a vaporizable material located within the oven chamber. The projection is shaped to correspond to a shape of the interior volume of the oven chamber. The vaporizer cover includes a concentrate oven disposed on the inner surface of the vaporizer cover. The concentrate oven extends from the inner surface of the vaporizer cover into the oven chamber. The concentrate oven is removably attached to the inner surface of the vaporizer cover. The concentrate oven includes a second conduction heater separate from the conduction heater used by the oven chamber. The vaporizer cover includes a concentrate oven cover disposed on the outer surface of the vaporizer cover, wherein the concentrate oven cover is slidably attached to the vaporizer cover to expose the concentrate oven. The concentrate oven is heated at least in part using the hybrid heating system of the vaporizer device. The concentrate oven includes one or more inlets to enable air flow into the concentrate oven. The vaporizer cover magnetically couples to the vaporizer body using at least one magnet. The oven chamber comprises one or more oven chamber inlets to allow for air to enter the oven chamber via the one or more oven chamber inlets. The air is pre-heated by the convection heater before entering the oven chamber via one or more inlets. The mouthpiece comprises an outlet through which aerosol is delivered to a user of the mouthpiece. The mouthpiece is tapered between two opposing long side portions of the mouthpiece. The vaporizer device comprises a cooling pathway formed between the oven chamber and the mouthpiece. The cooling pathway includes one or more features to create a turbulent path for an airflow from the oven chamber to the mouthpiece. The heating element is wrapped around at least a portion of the oven chamber.

In some embodiments, there is provided a vaporizer device including a mouthpiece; a hybrid heating system comprising a convection heater and a conduction heater; an oven chamber configured to contain a vaporizable material, wherein the vaporizable material is configured to be heated by convection, via air heated by the convection heater and by conduction, via contact with the oven chamber, wherein the oven chamber is configured to be heated by conduction using a heating element of the conduction heater, the heating element being in contact with the oven chamber; a vaporizer body comprising an outer shell which surrounds at least in part one or more internal components of the vaporizer body, the vaporizer body including a first end at which the mouthpiece is positioned and a second end opposite the first end, wherein the outer shell extends between the first end and the second end, the outer shell having two opposing long side portions and two opposing short side portions that define the outer shell, wherein the outer shell includes a recess exposing an interior of the vaporizer body, wherein the oven chamber is positioned in the vaporizer body and is exposed by the recess; and a vaporizer cover including an outer surface and an inner surface, wherein the outer surface is configured to face away from the vaporizer body, wherein the inner surface is opposite the outer surface and is configured to face towards an interior of the vaporizer body, and wherein the vaporizer cover includes a concentrate oven disposed on the inner surface of the vaporizer cover.

In some variations of the methods, systems, and non-transitory computer readable media, one or more of the following features can optionally be included in any feasible combination. The concentrate oven extends from the inner surface of the vaporizer cover into the oven chamber. The concentrate oven is removably attached to the inner surface of the vaporizer cover. The concentrate oven includes a second conduction heater separate from the conduction heater used by the oven chamber. The vaporizer device is configured to provide power to the concentrate oven rather than the oven chamber. In response to a mode change of the vaporizer or an automatic detection of the vaporizer cover including the concentrate oven being inserted into the recess, the vaporizer provides power to the concentrate oven.

In some embodiments, there is provided a vaporizer cover including a button disposed on an outer surface of the vaporizer cover; a concentrate oven disposed on an inner surface of the vaporizer cover; and one or more pins disposed on the inner surface of the vaporizer cover, wherein the vaporizer cover includes the outer surface and the inner surface, wherein the outer surface is configured to face away from a vaporizer body when the vaporizer body is coupled to the vaporizer body and the inner surface is opposite the outer surface and is configured to face towards an interior volume of the vaporizer body.

In some variations of the methods, systems, and non-transitory computer readable media, one or more of the following features can optionally be included in any feasible combination. The concentrate oven is configured to extend from the inner surface of the vaporizer cover into an oven chamber of a vaporizer device. The concentrate oven is removably attached to the inner surface of the vaporizer cover. The concentrate oven includes a second conduction heater configured to receive power via the one or more pins of the vaporizer cover. The vaporizer cover is shaped to fit in a recess of a vaporizer device. A user interface comprising one or more LEDs disposed on the outer surface. In response to a mode change of the vaporizer or an automatic detection of the vaporizer cover including the concentrate oven being inserted into a recess, the vaporizer cover receives power for the concentrate oven. One or more modes of the mode change include a first mode to power the concentrate oven and/or a second mode to power an oven chamber of a vaporizer device.

The details of one or more variations of the subject matter described herein are set forth in the accompanying drawings and the description below. Other features and advantages of the subject matter described herein will be apparent from the description and drawings, and from the claims. The claims that follow this disclosure are intended to define the scope of the protected subject matter.

### DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings:

FIG. 1 schematically illustrates a vaporizer body consistent with implementations of the current subject matter.

FIG. 2A illustrates an example of a vaporizer device consistent with implementations of the current subject matter.

FIG. 2B illustrates an example of a vaporizer device consistent with implementations of the current subject matter.

FIG. 2C illustrates an example of a vaporizer device consistent with implementations of the current subject matter.

FIG. 2D illustrates an example of a vaporizer device consistent with implementations of the current subject matter.

FIG. 2E is a schematic block diagram illustrating features of a vaporizer device consistent with implementations of the current subject matter.

FIGS. 3A and 3B schematically illustrate an example of a vaporizer device consistent with implementations of the current subject matter.

FIGS. 4A-4H illustrate examples of vaporizer devices, consistent with implementations of the current subject matter.

FIGS. 5A-5D illustrate examples of vaporizer devices, consistent with implementations of the current subject matter.

FIGS. 6A and 6B illustrate portions of example vaporizer devices, consistent with implementations of the current subject matter.

FIGS. 7A and 7B illustrate exploded views of an example of a vaporizer device, consistent with implementations of the current subject matter.

FIGS. 8A and 8B illustrate partial views of an example of a vaporizer device, consistent with implementations of the current subject matter.

FIG. 9 illustrates a partial cross-sectional view of an example of a vaporizer device including a convection heating system, consistent with implementations of the current subject matter.

FIGS. 10A and 10B illustrate cross-sectional views of an example vaporizer device and example airflow paths, consistent with implementations of the current subject matter.

FIGS. 11A-11C illustrate views of an example of a vaporizer device, consistent with implementations of the current subject matter.

FIGS. 12A-12K illustrate examples of vaporizer devices and covers, consistent with implementations of the current subject matter.

FIG. 13A illustrates an exploded view of an example of a vaporizer device, consistent with implementations of the current subject matter.

FIG. 13B illustrates an exploded view of a vaporizer cover including a concentrate oven, consistent with implementations of the current subject matter.

FIGS. 13C-13H illustrate different views of examples of vaporizer devices or vaporizer covers, consistent with implementations of the current subject matter.

FIGS. 14A-14C illustrate example modes of a vaporizer device, consistent with implementations of the current subject matter.

FIGS. 15A-15G illustrate examples of airflow paths for a concentrate cover and for a vaporizer device, consistent with implementations of the current subject matter.

FIG. 16 is an example chart depicting a method of generating an aerosol consistent with implementations of the current subject matter.

When practical, similar reference numbers denote similar structures, features, or elements.

### DETAILED DESCRIPTION

Implementations of the current subject matter include devices relating to vaporizing of one or more materials for inhalation by a user. The term “vaporizer” may be used generically in the following description and may refer to a vaporizer device, such as an electronic vaporizer. Vaporizers consistent with the current subject matter may be referred to by various terms such as inhalable aerosol devices, aerosolizers, vaporization devices, electronic vaping devices, electronic vaporizers, vape pens, etc. Examples of vaporizers consistent with implementations of the current subject matter include electronic vaporizers, electronic cigarettes, e-cigarettes, or the like. In general, such vaporizers are often portable, hand-held devices that heat a vaporizable material to provide an inhalable dose of the material. The vaporizer may include a heater configured to heat a vaporizable material which results in the production of one or more gas-phase components of the vaporizable material. A vaporizable material may include liquid and/or oil-type plant materials, or a semi-solid like a wax, or plant material such as leaves or flowers, either raw or processed. The gas-phase components of the vaporizable material may condense after being vaporized such that an aerosol is formed in a flowing air stream that is deliverable for inhalation by a user. The vaporizers may, in some implementations of the current subject matter, be particularly adapted for use with a plant material, such as a loose-leaf and/or full bud plant material (e.g., cannabis plant material), a concentrate material, and/or an oil-based vaporizable material, such as cannabis-derived, cannabidol-derived, and/or Tetrahydrocannabinol-derived oils although other types of vaporizable materials may be used as well.

Generally, a vaporizer device may store a vaporizable material, such as a plant material. When heating the plant material, the vaporizer device may either directly or indirectly heat the plant material (e.g., the vaporizable material), such as when the vaporizer device is activated. For example, the vaporizer device may heat the plant material, and after the plant material has been heated for a sufficient amount of time and/or to a desired temperature, a user may draw on the vaporizer device to inhale an aerosol including at least some of the vaporized plant material. In such devices, the generated aerosol may have an elevated temperature, as the aerosol travels directly from the storage compartment where the plant material is heated, through the mouthpiece of the vaporizer device, and to the user for inhalation. The heated aerosol may be harsh on the throat of the user when the user draws on the vaporizer device, resulting in an undesirable user experience. Additionally, some users may enjoy the experience of using a cannabis joint, which provides a large amount of smoke and high concentration of the vaporizable material entrained in the smoke for inhalation. Such a user experience may not be available in some vaporizer devices that are portable, small, and pocketable.

Vaporizer devices may heat the vaporizable material via conduction, convection, or radiation. However, conventional vaporizer devices, may, in some instances, heat the vaporizable material at slower speeds. As a result, a user may experience delays in heating the vaporizable material to a temperature at which the user may draw from the device. Further, in some instances, convection heating may be slower to heat the vaporizable material than conduction heating. For example, a heater may first be used to heat a container of the vaporizable material, which then heats the vaporizable material by contacting the vaporizable material. This process may quickly heat the vaporizable material. While conduction heating methods may heat the vaporizable material more quickly, convection heating methods may rapidly heat air that in turn heats the vaporizable material. Thus, conventional convection-based vaporizer devices may be slower to heat, and therefore less convenient for users. In both instances, some conventional vaporizer devices do not sufficiently provide an “on-demand” heating experience, in which the vaporizable material is rapidly heated for inhalation by the user. Rather, when using such vaporizer devices, there is an extended heat-up time to properly vaporizer the vaporizable material that is inconvenient, too slow, and/or the like, for users. Further, in some instances, the aerosol generated as a result of the vaporization of the vaporizable material may be further cooled before being delivered to the user, further delaying use of the vaporizer device by the user.

Moreover, in some vaporizer devices, the vaporizable material may be quickly heated by increasing a temperature of the heater. However, doing so may negatively impact the durability and/or properties of other components of the vaporizer device. For example, the high heater temperatures may cause other components to become damaged or otherwise degrade quickly, rendering the vaporizer device unusable or unsafe for use.

The portable vaporizer device consistent with implementations of the current subject matter may rapidly heat the vaporizable material. The vaporizer device described herein may sufficiently provide the on-demand heating experience. For example, the vaporizer device consistent with implementations of the current subject matter may include a hybrid heating system. The hybrid heating system may include a convention heating unit and/or a conduct heating unit. As a result, the vaporizable material may be rapidly heated by both conduction and convection heating, thereby rapidly heating the vaporizable material. In some implementations, the hybrid heating system may allow for selection of at least one or both of the conduction heating unit and the convection heating unit. In other words, the conduction heating unit or the convection heating unit (or both) can be selected, providing flexibility and improved options in heating the vaporizable material.

Additionally, or alternatively, the portable vaporizer device (which is consistent with implementations of the current subject matter) may include a unique cover. The cover includes a concentrate container (also referred to herein as a smart cover) into which concentrate material (an/or other materials) may be placed therein. The cover and the concentrate container are configured such that when the cover and concentrate container are coupled to the vaporizer device, the concentrate container is within the oven chamber of the vaporizer. Alternatively, or additionally, the concentrate container may be heated at least in part by a heater that is separate from the heater used by the oven chamber. For example, the concentrate container may have its own conduction heater in contact with the concentrate container (also referred to as “concentrate oven”). Alternatively, or additionally, the cover including the concentrate container may be positioned on a long side of the vaporizer device, rather than at the bottom of the vaporizer or the top of the vaporizer (where the mouthpiece is located).

Additionally, and/or alternatively, the vaporizer device consistent with implementations of the current subject matter includes insulation that helps to cool the heater (e.g., the convection heater), such as when the heater is heated to high temperatures when rapidly heating the air and/or vaporizable material within the vaporizer device. Such insulation helps to prevent damage in other components of the vaporizer device.

Additionally, and/or alternatively, the portable vaporizer device consistent with implementations of the current subject matter may quickly and efficiently cool the generated aerosol before the aerosol is delivered to the user. For example, the vaporizer device includes a cover having a projection that extends into an oven chamber of the vaporizer device. With the generated aerosol, the projection creates a turbulent airflow to quickly and efficiently cool the generated aerosol. The generated aerosol may also be delivered along a cooling pathway that extends along a length of the vaporizer device before the aerosol is delivered to the user to further cool the aerosol. Such configurations provide smooth and cooled vapor that is less harsh on the user (e.g., the throat of the user), helps to limit or reduce the amount of heated vapor the user inhales during a draw on the vaporizer device, and/or provides a more flavorful aerosol. Such configurations may also provide a greater amount of aerosol with higher concentrations of the vaporized vaporizable material. Thus, the vaporizer device consistent with implementations of the current subject matter may provide a user experience that is similar to and/or improved upon the experience for the user using a cannabis joint.

Aspects of the current subject matter relate to a vaporizer device that vaporizes a vaporizable material, such as loose-leaf and/or full bud plant material (e.g., cannabis plant material), a concentrate material (e.g., wax, shatter, budder, butane hash oil, and the like), and/or one or more liquids, such as oils, extracts, aqueous or other solutions, etc., of one or more substances, among other vaporizable materials as described herein that may be desirably provided in the form of an inhalable aerosol.

Aspects of the current subject matter relate to heating (e.g., via the hybrid heating system) of the vaporizable material stored in the vaporizer device (e.g., a vaporizer cartridge, a vaporizer body, and/or the like). As heating of a vaporizable material is directly correlated with aerosol production, adequate heating of the vaporizable material aids in providing a user a consistent and desired experience. A greater variability in the heating of the vaporizable material when a user puffs on the vaporizer device results in a greater variability in the amount of aerosol produced by the vaporizer device, which may lead to an inconsistent, unsatisfying, and/or undesirable user experience. Greater variability in the heating of the vaporizable material may also make it more difficult to control and/or monitor a precise amount of generated aerosol. Moreover, variability in aerosol production correlates to variability in dosage, which may be of particular concern in medicinal applications. Aspects of the current subject matter provide for improved heating of the vaporizable material for improved aerosol production.

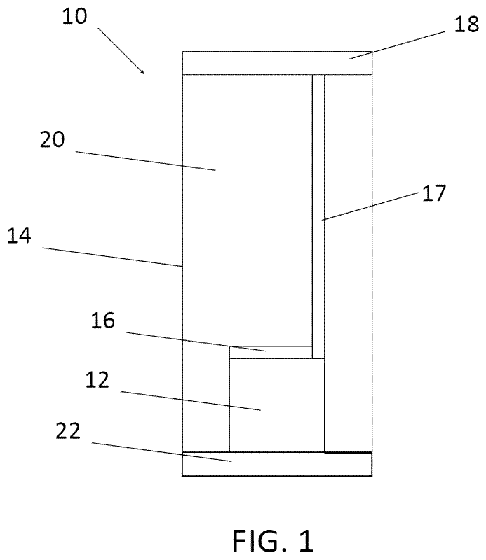

FIG. 1 schematically illustrates an example of a vaporizer device 10, consistent with implementations of the current subject matter. The vaporizer device 10 includes a vessel 12 contained within a housing or vaporizer body 14, and further includes a heating element 16 that is configured to elevate a temperature of the vessel 12 and/or within the vessel 12 to a level and/or range that is suitable for vaporizing a vaporizable material. The vessel 12 may be positioned within a cavity of the vaporizer body 14 of the vaporizer device 10. The vessel 12 and/or the cavity may be positioned at an end of the vaporizer device 10 and/or along a side of the vaporizer device 10.

As shown in FIG. 1, the vaporizer device 10 may include or be coupled with an oven. The oven may form at least a part of a heating element 16. For example, the oven may be heated, may include the heating element 16, and/or may be coupled to the heating element 16. In some implementations, the oven is defined by the vessel 12. The oven may be configured to contact at least a portion of the vaporizable material to heat the vaporizable material.

In some implementations, the vaporizer device 10 includes a lid 22 that closes and/or fits over at least a portion of an open end of the vaporizer body 14 of the vaporizer device that includes the vessel 12, forming an air chamber. The lid 22 is configured to enclose the vessel 12, such as when the vaporizable material is positioned within the vessel 12. When the heating element 16 is activated, the vaporizer device 10 heats and vaporizes the vaporizable material when the vaporizable material is placed within the vessel 12.

Heat transfer occurs between the vessel 12, the heating element 16, and the vaporizable material contained therein. For example, the heater may include a conduction, a convection, and/or an induction heating system. In particular as described herein, the heater may include a hybrid heating system including a conduction heater and a convection heater. Thus, the heater may rapidly heat the vaporizable material stored within the vessel 12 via conduction and convection. When the heater heats the vaporizable material, at least a portion of the vaporizable material may rapidly vaporize and mix with air in the air chamber to form an aerosol. The aerosol travels through an air path 17 through the vaporizer body 14 and exits from the vaporizer body 14 through a mouthpiece 18. The mouthpiece 18 is configured to enable a user to draw, for example through inhalation, the aerosol from the vaporizer device. The vaporizer device 10 may have an elongated cylindrical shape, an elongated flattened shape, and/or the like. While FIG. 1 illustrates an example of the vaporizer device 10 with the vessel 12 positioned at a distal end of the vaporizer device 10 and the mouthpiece 18 positioned at a proximal end of the vaporizer device 10 opposite the distal end, other configurations are contemplated, such that the vessel 12 may be positioned at a same end of the vaporizer body 14 as the mouthpiece 18, and/or the vessel 12 may be positioned at another portion of the vaporizer body 14, such as between the proximal end and the distal end of the vaporizer body 14.

The vaporizer body 14 and/or the lid 22 may include one or more apertures (e.g., inlets, holes, opening, gaps, spaces, cracks, slits, and/or a like) configured to allow air to enter into the vessel 12 from, for example, outside of the vaporizer device 10. A user inhaling from the mouthpiece 18 of the vaporizer device 10 causes an intake of air into the vessel 12. The incoming air mixes with the vapor generated by the vaporization of vaporizable material. The resulting airflow carries the aerosol through the air path 17 to the mouthpiece 18 where the aerosol is delivered to the user.

The lid 22 and/or vaporizer body 14 may include one or more mechanisms, for example, snaps, latches, grooves, threading, magnets, clips, quick connect, sliding mechanisms, quarter turn release, friction fit, and the like, configured to position and/or secure the lid 22 against the vaporizer body 14.

An outer shell 20 (which may surround all or a portion of the housing 14) or cover of the vaporizer device 10 may be made of various types of materials, including for example aluminum (e.g., AL6063, AL6061), stainless steel, glass, ceramic, titanium, plastic (e.g., Acrylonitrile Butadiene Styrene (ABS), Nylon, Polycarbonate (PC), Polyether Sulfone (PESU), and the like), fiberglass, carbon fiber, and any hard, durable material.

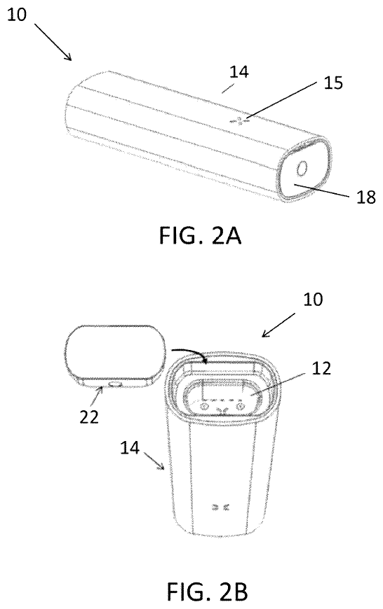

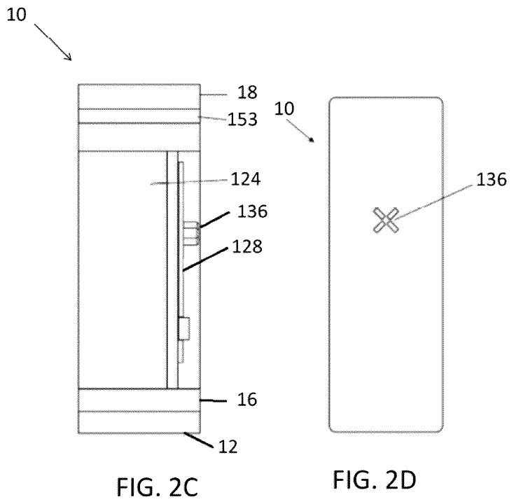

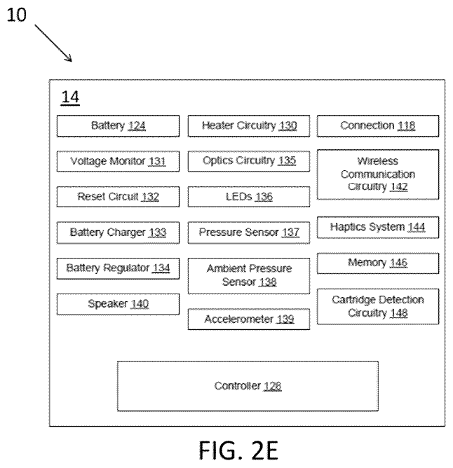

FIG. 2A shows a side perspective view of an exemplary vaporizer device 10 consistent with implementations of the current subject matter. FIG. 2B illustrates a bottom perspective view of the vaporizer device 10 consistent with implementations of the current subject matter. As shown in FIGS. 2A and 2B, the vaporizer device 10 includes a vaporizer body 14, a mouthpiece 18, and a lid 22. The lid 22 may be removed (see FIG. 2B) from the vaporizer body 14, exposing the vessel 12, which as described herein may define an oven or vaporization chamber where the vaporizable material stored within the vessel is heated to generate an aerosol. The mouthpiece 18 may be flat and have a side opening region through which the aerosol may exit. Additionally and/or alternatively, the mouthpiece may extend from the vaporizer body 14 and include an opening through which the vapor may exit.

Further the mouthpiece 18 may be made of any appropriate material, including both conductive and non-conductive materials. For example, the mouthpiece 18 may be polymeric (e.g., silicone), plastic, metal, ceramic, and the like. The vaporizer body 14 can be a single extruded aluminum outer body. Further, the mouthpiece 18 can be removable from the vaporizer body 14. The vaporizer device 10 may further include an indicator 136, such as an LED indicator. In some implementations, the indicator 136 can indicate a temperature status and/or activation status of the device 10.

FIGS. 2C and 2D schematically illustrates an example of the vaporizer device 10 consistent with implementations of the current subject matter. A shown in FIGS. 2C and 2D, the vaporizer device 10 may include the mouthpiece 18 adjacent to which a capacitive sensor 153 is attached, which may be used to sense contact with the user (e.g., the users lip or mouth) in contact with the mouthpiece. Although FIG. 2C depicts a capacitive sensor, other types of sensors may detect the contact of the lip, the user hand when making contact with the vaporizer, and/or inhalation or puff (e.g., via a pressure sensor or a microphone sensor). This input (e.g., from a sensor or capacitive sensor) may be connected to a controller 128 (see FIG. 2E). A battery 124 may provide power to the vaporizer device 10 and may be rechargeable or disposable. The heating element 16 may be controlled by the controller 128. In some variations an oven (e.g., the vessel 12) is coupled to the heating element 16 for vaporizing the vaporizable material, as described herein.

FIG. 2E is a schematic block diagram illustrating components of the vaporizer device 10 a vaporizer body or housing 14 consistent with implementations of the current subject matter. Included in the vaporizer body 14 is a controller 128 that includes at least one processor and/or at least one memory configured to control and manage various operations among the components of the vaporizer device 10 described herein.

Heater control circuitry 130 of the vaporizer body 14 controls the heating element 16. The heating element 16 may generate heat to provide vaporization of the vaporizable material. For example, the heating element 16 may include a heating coil, resistive wire (e.g., a resistive heater), inductive heating system, conduction heating system, convection heating system, and/or other heating element in thermal contact with the vaporizable material, as described in further detail below. For example, the heating element 16 may include a first heating element of the convection heating system and a second heating element of the conduction heating system, as part of the hybrid heating system described herein.

A battery 124 is included in the vaporizer body 14, and the controller 128 may control and/or communicate with a voltage monitor 131 which includes circuitry configured to monitor the battery voltage, a reset circuit 132 configured to reset (e.g., shut down the vaporizer device 10 and/or restart the vaporizer device 10 in a certain state), a battery charger 133, and a battery regulator 134 (which may regulate the battery output, regulate charging/discharging of the battery, and provide alerts to indicate when the battery charge is low, etc.). The controller 128 may regulate the power flow (e.g., an amount or current and/or a voltage amount) to control a temperature at which the heating element 16 heats the vaporizable material contained in the vessel 12.

The controller 128 may control and/or communicate with optics circuitry 135 (which controls and/or communicates with one or more indicators, such as LEDs 136 which can provide user interface output indications), a pressure sensor 137, an ambient pressure sensor 138, an accelerometer 139, and/or a speaker 140 configured to generate sound or other feedback to a user.

Alternatively, or additionally, a pressure sensor 137 may be configured to sense a user drawing (e.g., inhaling) on the mouthpiece 18 and activate the heater control circuitry 130 of the vaporizer body 14 to accordingly control the heating element 16. In this way, the amount of current supplied to the heating element 16 may be varied according the user's draw (e.g., additional current may be supplied during a draw, but reduced when there is not a draw taking place). The ambient pressure sensor 138 may be included for atmospheric reference to reduce sensitivity to ambient pressure changes and may be utilized to reduce false positives potentially detected by the pressure sensor 137 when measuring draws from the mouthpiece 18.

Alternatively, or additionally, the accelerometer 139 (and/or other motion sensors, capacitive sensors, flow sensors, strain gauge(s), or the like) may be used to detect user handling and interaction, for example, to detect movement of the vaporizer body 14 (such as, for example, tapping, rolling, and/or any other deliberate movement associated with the vaporizer body 14). The detected movements may be interpreted by the controller 128 as one or more predefined user commands. For example, one particular movement may be a user command to gradually increase the temperature of the heating element 16 as the user intends to begin using the vaporizer device 10.

Alternatively, or additionally the vaporizer body 14, as shown in FIG. 2C for example, includes wireless communication circuitry 142 that is connected to and/or controlled by the controller 128. The wireless communication circuitry 142 may include a near-field communication (NFC) antenna that is configured to read from and/or write to a tag, and the like. Alternatively or additionally, the wireless communication circuitry 142 may be configured to automatically detect the vaporizable material when the vaporizable material is inserted into the vaporizer body 14.

Alternatively, or additionally, the wireless communication circuitry 142 may include additional components including circuitry for other communication technology modes, such as Bluetooth circuitry, Bluetooth Low Energy circuitry, Wi-Fi circuitry, cellular (e.g., LTE, 4G, and/or 5G) circuitry, and associated circuitry (e.g., control circuitry), for communication with other devices. For example, the vaporizer body 14 may be configured to wirelessly communicate with a remote processor (e.g., a smartphone, a tablet, a computer, wearable electronics, a cloud server, and/or processor based devices) through the wireless communication circuitry 142, and the vaporizer body 14 may through this communication receive information including control information (e.g., for setting temperature, resetting a dose counter, etc.) from and/or transmit output information (e.g., dose information, operational information, error information, temperature setting information, heater information, charge/battery information, etc.) to one or more of the remote processors.

Alternatively, or additionally, the vaporizer body 14 may include a haptics system 144, such as an actuator, a linear resonant actuator (LRA), an eccentric rotating mass (ERM) motor, or the like that provide haptic feedback such as a vibration as a “find my device” feature or as a control or other type of user feedback signal. For example, using an app running on a user device (such as, for example, a user device), a user may indicate that he/she cannot locate his/her vaporizer device 10. Through communication via the wireless communication circuitry 142, the controller 128 sends a signal to the haptics system 144, instructing the haptics system 144 to provide haptic feedback (e.g., a vibration). The controller 128 may additionally or alternatively provide a signal to the speaker 140 to emit a sound or series of sounds. The haptics system 144 and/or speaker 140 may also provide control and usage feedback to the user of the vaporizer device 10; for example, providing haptic and/or audio feedback when a particular amount of a vaporizable material has been used or when a period of time since last use has elapsed. Alternatively, or additionally, haptic and/or audio feedback may be provided as a user cycles through various settings of the vaporizer device 10. Alternatively, or additionally, the haptics system 144 and/or speaker 140 may signal when a certain amount of battery power is left (e.g., a low battery warning and recharge needed warning) and/or when a certain amount of vaporizable material remains. Alternatively, or additionally, the haptics system 144 and/or speaker 140 may also provide usage feedback and/or control of the configuration of the vaporizer device 10 (e.g., allowing the change of a configuration, such as target heating rate, heating rate, etc.).

Alternatively, or additionally, the vaporizer body 14 also includes the connection (e.g., USB-C connection, micro-USB connection, and/or other types of connectors) 118 for coupling the vaporizer body 14 to a charger to enable charging the internal battery 124. Alternatively or additionally, electrical inductive charging (also referred to as wireless charging) may be used, in which case the vaporizer body 14 would include inductive charging circuitry to enable charging. The connectors 118 at FIG. 2C may also be used for a data connection between a computing device and the controller 128, which may facilitate development activities such as, for example, programming and debugging.

Alternatively, or additionally, the vaporizer body 14 may also include a memory 146 that is part of the controller 128 or is in communication with the controller 128. The memory 146 may include volatile and/or non-volatile memory or provide data storage. In some implementations, the memory 146 may include 8 Mbit of flash memory, although the memory is not limited to this and other types of memory may be implemented as well.

The vaporizer device 10 also includes a vaporizing assembly of vapor-generating components. The vapor-generating components may include the heating element 16 configured to heat the vaporizable material, such as via the hybrid heating system described herein, to a sufficient temperature that it may vaporize. The vapor-generating components may be arranged as an atomizer or cartomizer or oven. The vapor may be released to a vaporization chamber where the gas phase vapor may condense, forming an aerosol cloud having typical liquid vapor particles with particles having a diameter of average mass of approximately 0.1 micron or greater. In some cases, the diameter of average mass may be approximately 0.1-1 micron.

The heating element 16 of the vaporizing assembly may cause the vaporizable material to be converted from a condensed form (e.g., a solid, a liquid, a solution, a gel, a concentrate, a suspension, a part of an at least partially unprocessed plant material, etc.) to the gas phase. After conversion of the vaporizable material to the gas phase, and depending on the type of vaporizer, the physical and chemical properties of the vaporizable material, and/or other factors, at least some of the gas-phase vaporizable material may condense to form particulate matter in at least a partial local equilibrium with the gas phase as part of an aerosol, which may form some or all of an inhalable dose provided by the vaporizer device 10 for a given puff or draw on the vaporizer device 10. It will be understood that the interplay between gas and condensed phases in an aerosol generated by a vaporizer may be complex and dynamic, as factors such as ambient temperature, relative humidity, chemistry, flow conditions in airflow paths (both inside the vaporizer and in the airways of a human or other animal), mixing of the gas-phase or aerosol-phase vaporizable material with other air streams, etc., may affect one or more physical parameters of an aerosol.

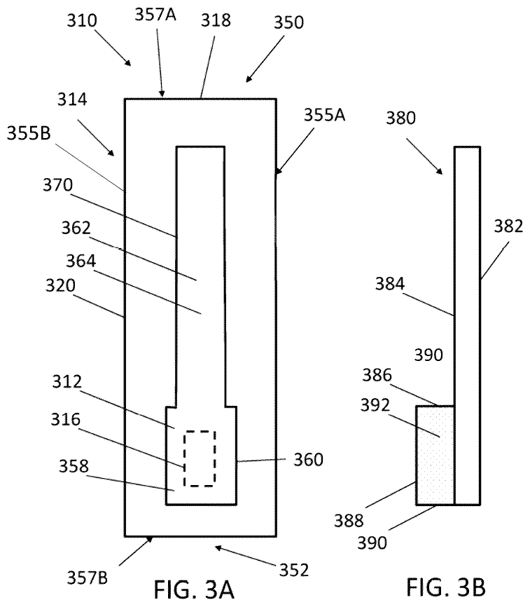

FIG. 3A and FIG. 3B schematically illustrate a vaporizer device 310, consistent with implementations of the current subject matter. The vaporizer device 310 may be the same as or similar to the vaporizer device 10. For example, one or more of the components of the vaporizer device 10 may be used with and/or instead of one or more of the components of the vaporizer device 310 and/or one or more of the components of the vaporizer device 310 may be used with and/or instead of one or more of the components of the vaporizer device 10. As described herein, the vaporizer device 310 may be a portable vaporizer device. The vaporizer device 310 may include a vaporizer body or housing 314 and a cover 380.

FIG. 3A schematically illustrates the vaporizer body 314, consistent with implementations of the current subject matter. As shown in FIG. 3A, the vaporizer body 314 includes a vessel or oven chamber 312, which is configured to hold a vaporizable material, a hybrid heating system 316, which is configured to cause vaporization of the vaporizable material to generate an aerosol, and a mouthpiece 318, which is configured to deliver the generated aerosol to a user. The hybrid heating system 316 may be coupled to and/or form a part of the oven chamber 312.

The hybrid heating system 316 consistent with implementations of the current subject matter may include a conduction heating unit and a convection heating unit. For example, air entering the vaporizer device 310 may be heated, by the convection heating unit, and enter the oven chamber 312, wherein the heated air assists in heating the vaporizable material to vaporize the vaporizable material. In other words, the convection heating unit heats the air before the air passes into the oven chamber 312. The heated air may heat the vaporizable material stored within the oven chamber 312 to cause vaporization of the vaporizable material. Additionally, and/or alternatively, as part of the conduction heating unit, heat transfer occurs between a heating element coupled to the oven chamber 312, the oven chamber 312, and the vaporizable material contained within and in contact with the oven chamber 312. For example, the heating element may be wrapped around the oven chamber 312, contact a base of the oven chamber 312, or contact the oven chamber 312 in another manner. In such configurations, the heating element includes a conduction heater configured to heat the oven chamber 312 in contact with the vaporizable material to cause vaporization of the vaporizable material. Thus, the hybrid heating system 316 may heat the vaporizable material stored within the oven chamber 312 via conduction and/or convection.

The oven chamber 312 can be positioned on a side of the vaporizer body 314. For example, the vaporizer body 314 may include a first end 350 at which a mouthpiece 318 is positioned, and a second end 352 opposite the first end 350. The vaporizer body 314 also includes an outer shell 320 that surrounds the internal components of the vaporizer body 314. The outer shell 320 may extend between the first end 350 and the second end 352. The outer shell 320 may include two opposing long side portions 355A, 355B, and two opposing short side portions 357A, 357B. The two opposing long side portions 355A, 355B and the two opposing short side portions 357A, 357B are integrally formed to define the outer shell 320. Each of the two opposing short side portions 357A, 357B extend between the two opposing long side portions 355A, 355B. The oven chamber 312 may be positioned in one of the two opposing long side portions 355A, 355B, such as in the long side portion 355B.

The oven chamber 312 may be positioned closer to one end of the vaporizer body 314 than to the opposite end of the vaporizer body 314. For example, the oven chamber 312 may be positioned closer to the second end 352 than to the first end 350, where the mouthpiece 318 is located. In the example of FIG. 3A, the oven chamber is positioned in the lower half of the vaporizer body. Alternatively, the oven chamber may be positioned in the lower third of the vaporizer body.

The oven chamber 312 may include an oven base 358 and one or more side walls 360 extending from the oven base 358. The one or more side walls 360 may extend between the oven base 358 and the outer shell 320. The one or more side walls 360 and the oven base 358 may define an interior volume in which the vaporizable material is positioned. The one or more side walls 360 may wrap around a perimeter of the oven chamber 312. The one or more side walls 360 may be perpendicular to the oven base 358 and/or to the outer shell 320. In some implementations, the one or more side walls 360 are flared outwardly from the oven base 358 towards the outer shell 320. Such configurations may allow for easier loading of the oven chamber 312 with the vaporizable material.

The mouthpiece 318 may be positioned at the first end 350 of the vaporizer body 314. The generated aerosol may pass from the oven chamber 312 along an air path to the mouthpiece 318 positioned at and/or towards an opposite end of the vaporizer body 314 to allow the generated aerosol to cool before being delivered to the user.

For example, the vaporizer device 310 may include a cooling pathway 362 formed between the oven chamber 312 and the mouthpiece 318. The cooling pathway 362 may define a flat channel formed between the oven chamber 312 and the mouthpiece 318. In some implementations, the cooling pathway 362 includes one or more features to create a turbulent path for the airflow from the oven chamber 312 to allow for sufficient cooling. Such a turbulent path may include a zig-zag path, a path with various bumps and/or projections, or other configurations, to allow for the quick exchange of heat away from the heated aerosol.

In some implementations, the vaporizer body 314 includes a recess 370. The recess 370 extends along a length of the vaporizer body 314. For example, the recess 370 may be positioned along one of the two opposing long side portions 355A, 355B of the outer shell 320. As shown in FIG. 3A, the recess 370 is positioned in the long side portion 355B of the outer shell 320. The recess 370 extends from the outer shell 320 towards an interior of the vaporizer body 314. The oven chamber 312 and the cooling pathway 362 may be positioned within the recess 370. As described herein, the oven chamber 312 includes the oven base 358. The cooling pathway 362 may include a cooling base 364 forming a part of the flat channel of the cooling pathway 362. The oven base 358 may be positioned inwardly from the outer shell 320 of the vaporizer body 314 at a first distance. The cooling base 364 may be positioned inwardly from the outer shell 320 at a second distance. The first distance may be greater than the second distance. In other words, the oven base 358 may be positioned farther towards an interior of the vaporizer body 314 than the cooling pathway 362, relative to the outer shell 320. The recess 370 may be open such that the oven chamber 312 and the cooling pathway 362 are exposed. For example, the recess 370 may be uncovered when the vaporizable material is deposited into the oven chamber 312 for heating.

FIG. 3B schematically illustrates a cover 380 (also referred to as a vaporizer cover herein), consistent with implementations of the current subject matter. The cover 380 is removable from the vaporizer body 314. The cover 380 is configured to enclose the oven chamber 312 and the cooling pathway 362. Additionally, and/or alternatively, the cover 380 is sized and shaped to enclose the recess 370. Additionally, and/or alternatively, the cover 380 encloses the recess 370 and is in parallel to the long sides 355A-B. The cover 380 includes an outer surface 382 configured to face away from the vaporizer body 314 and an inner surface 384 opposite the outer surface 382 and configured to face towards an interior of the vaporizer body 314, such as face towards the oven chamber 312, the cooling pathway 362, and/or the recess 370.

In some embodiments, the cover 380 also includes a projection 386. The projection 386 may be solid material, such that the projection 386 compresses the vaporizable material within the oven chamber 312. This allows for improved, consistent, and/or even heating of the vaporizable material within the oven chamber 312. Additionally, and/or alternatively, the projection 385 may include one or more tortuous features 392 that create turbulent airflow of the generated aerosol passing from the oven chamber 312 to the cooling pathway 362 to cool the generated aerosol. Alternatively, or additionally, the projection may comprise a concentrate oven configured to heat concentrate placed inside the concentrate oven, in accordance with some embodiments. In some embodiments, the vaporizer device may be used with two different types of vaporizer covers. For example, a first type of vaporizer cover may include a projection, and a second type of vaporizer cover may include a concentrate oven. The vaporizer device may detect whether the second type of vaporizer cover is being used, and, if so, power the concentrate oven to heat the material therein.

The projection 386 is configured to extend from the inner surface 384. For example, the projection 386 is configured to extend from the inner surface 384 into an interior volume of the oven chamber 312. The projection 386 may be positioned along a portion of the cover 380 that is less than one half a length of the cover 380. In other words, the projection 386 may be positioned closer to one end of the cover 380 than to the opposite end of the cover 380. For example, the projection 386 is positioned at one end of the cover 380. The positioning of the projection 386 corresponds to the position of the oven chamber 312 in the vaporizer body 314.

The projection 386 has a shape that corresponds to a shape of the oven chamber 312. For example, the projection 386 includes a projection base portion 388 and a projection sidewall portion 390. The projection sidewall portion 390 extends from the projection base portion 388 towards the inner surface 384 of the cover 380. In other words, the projection sidewall portion 390 extends inwardly and away from the inner surface 384 towards the projection base portion 388. The projection sidewall portion 390 corresponds to the side wall 360 of the oven chamber 312. In the example of FIG. 3B, the sidewalls 390 are perpendicular to the inner surface 384 and the base 388 is parallel to the inner surface, so as to form a rectangular projection; however, the projection may take other forms so long as the projection can extend into the size and shape of the oven chamber.

In some implementations, the one or more tortuous features 392 may be formed in the projection 386. For example, the one or more tortuous features 392 are formed in the projection base portion 388 and/or the projection sidewall portion 390. The one or more tortuous features 392 include one or more (e.g., one or a plurality) perforations, slits, openings, and/or the like. The one or more tortuous features 392 can include an array of tortuous features 392. The one or more tortuous features 392 create turbulent airflow of the generated aerosol passing from the oven chamber 312 to the cooling pathway 362 to cool the generated aerosol. For example, the one or more tortuous features 392 allow the generated aerosol to pass from the oven chamber 312, through the tortuous features 392, into an interior volume of the projection 386. The one or more tortuous features 392 creates turbulence in the air, which further mixes and cools within the interior volume of the projection 386. The one or more tortuous features 392 may be evenly spaced from one another and/or may have variable spacing. The spacing of the one or more tortuous features 392 may be optimally spaced, shaped, and/or sized to create turbulent airflow of the generated aerosol to cool the generated aerosol. In some implementations, the projection 386 does not include the tortuous features 392.

In some implementations, air may pass through at least one inlet (not shown) into the vaporizer device and into the oven chamber 312 where the vaporizable material is stored and heated. Heat transfer occurs between the heated air and the vaporizable material, and/or between the heated oven chamber 312 and the vaporizable material contained within the oven chamber 312 to heat the vaporizable material. When the vaporizable material is heated, such as by both conduction and convection as part of the hybrid heating system, at least a portion of the vaporizable material may rapidly vaporize and mix with air in the oven chamber 312 to form an aerosol. The aerosol travels from the oven chamber 312 to create a turbulent airflow. The turbulent airflow mixes within the interior volume of the projection 386 to cool (e.g., reduce a temperature) the generated airflow. Additionally, and/or alternatively the aerosol may then pass to the cooling pathway 362. The cooling pathway 362 may be formed within in an interior volume of the cover 380 and/or along a portion of the recess 370, as described herein, for further cooling of the aerosol. The aerosol is then delivered to the mouthpiece 318 where the aerosol exits the vaporizer body 314. The mouthpiece 318 is configured to enable a user to draw, for example through inhalation, the aerosol from the vaporizer device.

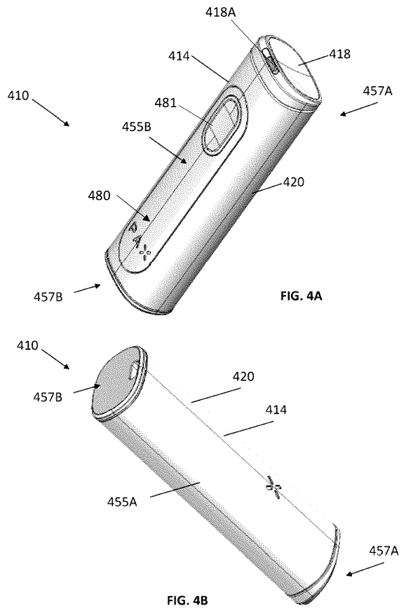

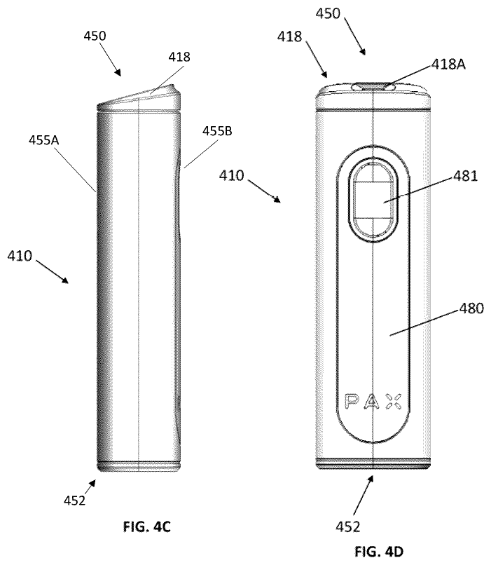

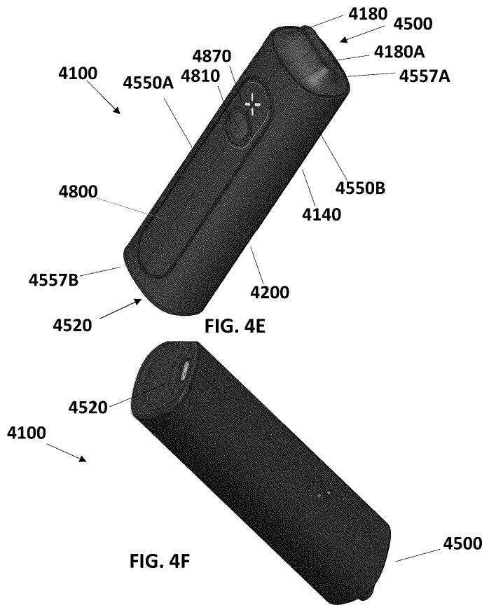

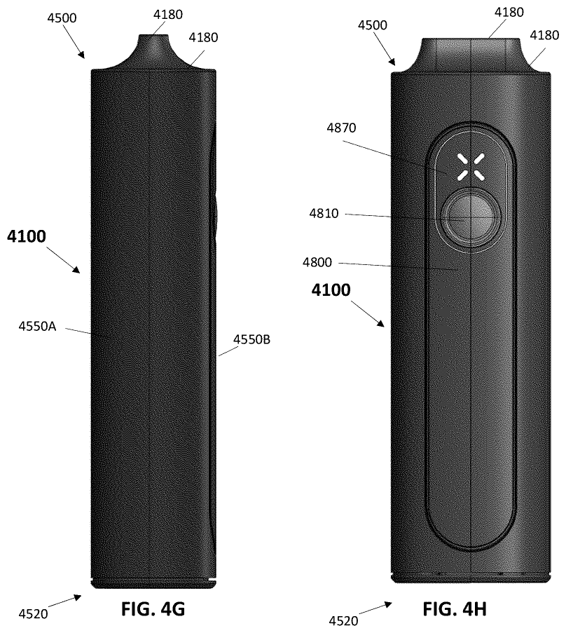

FIGS. 4A-4D illustrate an example of a vaporizer device 410, which may include the same or similar features and/or components as the vaporizer device 10 and/or 310 and may include one or more components that are the same as or may be interchanged with one or more components of the vaporizer device 10 and 310, consistent with implementations of the current subject matter. As shown in FIGS. 4A-4D, the vaporizer device 410 includes a vaporizer body 414. The vaporizer body 314 includes an outer shell 420, which includes two opposing long side portions 455A, 455B, two opposing short side portions 457A, 457B, a first end 450, and a second end 452.

Referring to FIGS. 4A-4D, the vaporizer device 410 includes a mouthpiece 418, consistent with implementations of the current subject matter. The mouthpiece 418 is positioned at the first end 450, which is opposite the second end 452. The mouthpiece 418 may include an outlet 418A, through which the generated aerosol may be delivered to the user. The mouthpiece 418 may be tapered or have a tapered side profile. For example, the mouthpiece 418 may be tapered between the opposing long side portions 455A, 455B.

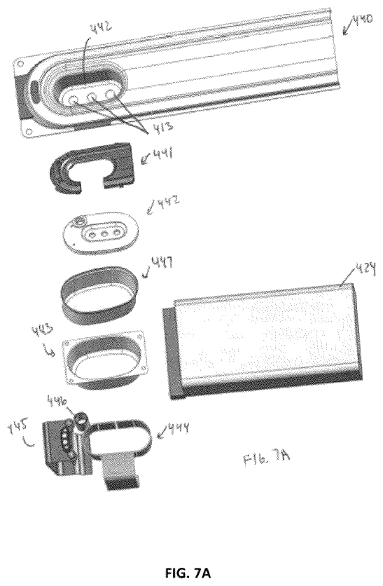

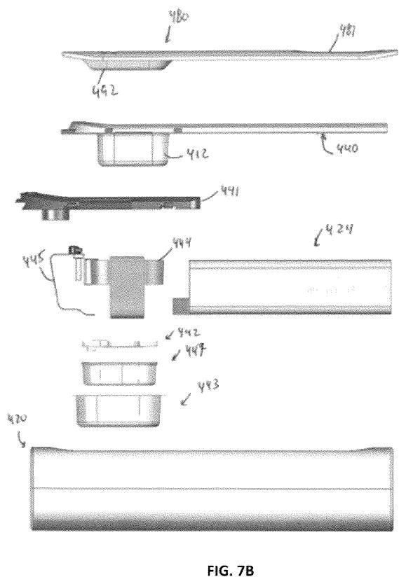

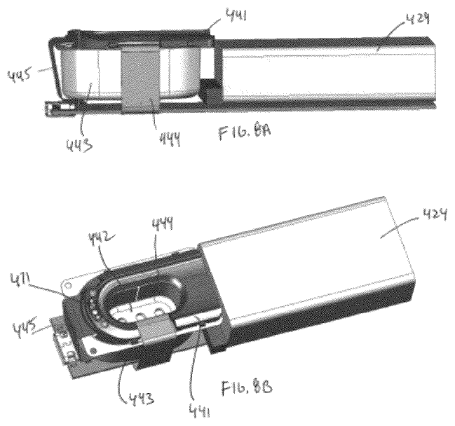

Again referring to FIGS. 4A-4D, the vaporizer device 410 includes a cover 480. The cover 480 may be magnetically coupled to the vaporizer body 414. The cover 480 may include a button 481 (e.g., a receiver, a toggle, and/or the like), which may be manipulated to couple or decouple the cover 480 from the vaporizer body 414, facing away from the vaporizer body 414. The cover 480 may include a projection 492 which faces the vaporizer body 414 (as noted above with respect to FIG. 3B as well as described below with for example FIG. 7B).

FIGS. 4E-4H illustrate another example of a vaporizer device 4100, which may include some of the same or similar features and/or components as the vaporizer device 10, 310, and 410 and may include one or more components that are the same (or similar) as or may be interchanged with one or more components of the vaporizer device 10, 310, and 410 consistent with implementations of the current subject matter. The vaporizer device 4100 includes a vaporizer body 4140. The vaporizer body 4140 includes an outer shell 4200, which includes two opposing long side portions 4550A, 4550B, two opposing short side portions 4570A, 4570B, a first end 4500, and a second end 4520.

The vaporizer device 4100 includes a mouthpiece 4180, consistent with implementations of the current subject matter. The mouthpiece 4180 is positioned at the first end 4500, opposite the second end 4520. The mouthpiece 4180 may include an outlet 4180A, through which the generated aerosol may be delivered to the user. The mouthpiece 4180 may be tapered or have a tapered side profile. For example, the mouthpiece 4180 may be tapered between the opposing long side portions 4550A, 4550B.

The vaporizer device 4100 includes a cover 4800. At least some of the aspects of the cover 4800 may be the same (or similar) to the cover 380 noted above with respect to FIG. 3B. For example, the cover 48000 may be magnetically coupled to the vaporizer body 4140. Alternatively, or additionally, the cover 4800 may include a button 4810 (e.g., a receiver, toggle, and/or the like), which may be manipulated to couple or decouple the cover 4800 from the vaporizer body 4140. Alternatively, or additionally, the vaporizer device 4100 (or the cover 4800) may include one or more LEDs 4870, which can be used to provide indications to a user (e.g., an indication of power, mode changes, heater temperature, and/or the like). The cover 4800 may include a projection 392.

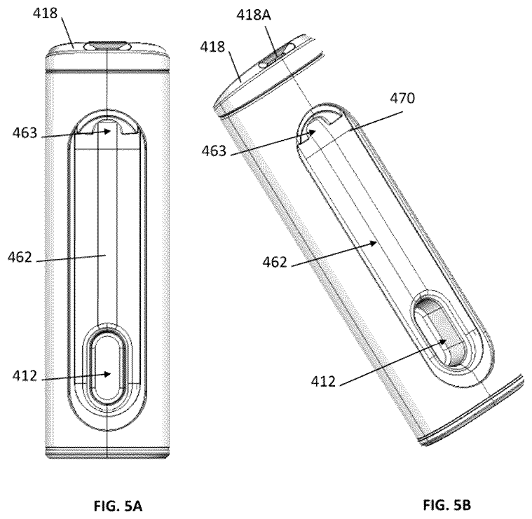

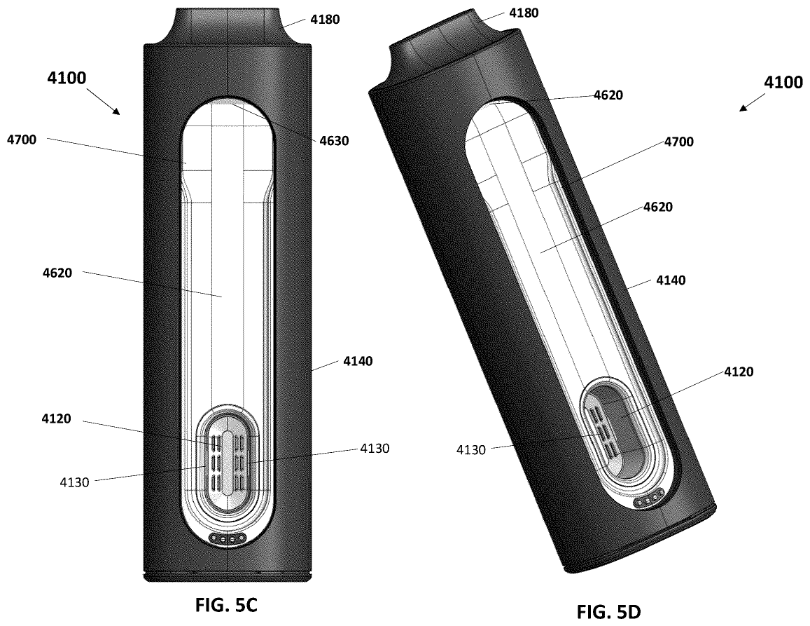

FIGS. 5A and 5B illustrate an example of the vaporizer device 410, in which the cover 480 has been removed (or otherwise detached) from the vaporizer body 414. As shown in FIGS. 5A and 5B, removal of the cover 480 (see, e.g., FIG. 4A) exposes an oven chamber 412 and a cooling pathway 462 of the vaporizer body 414. Consistent with implementations of the current subject matter, the oven chamber 412 and the cooling pathway 462 may be positioned within a recess 470 formed in the vaporizer body 414. As shown, the cooling pathway 462 extends between the oven chamber 412 and an outlet 463 at an end (e.g., an opposite end) of the cooling pathway 462).

FIGS. 5C and 5D further illustrate the vaporizer device 4100, in which the cover 4800 has been removed (or otherwise detached) from the vaporizer body 4140. As shown in FIGS. 5C and 5D, the removal of the cover 4800 exposes an oven chamber 4120 and a cooling pathway 4620 of the vaporizer body 4140. Consistent with implementations of the current subject matter, the oven chamber 4120 and the cooling pathway 4620 may be positioned within a recess 4700 formed in the vaporizer body 4140. As shown, the cooling pathway 4620 extends between the oven chamber 4120 and an outlet 4630 at an end (e.g., an opposite end) of the cooling pathway 4620). In the example of FIG. 5C, the oven chamber includes one or more inlets 4130. When a user inhales at the mouth piece, air enters the vaporizer body and enters the oven chamber 4120 via the inlets 4130, where the vaporizable material is vaporized to generate an aerosol carried via the cooling pathway 4620 to the mouthpiece 4180. Although FIG. 5C depicts 12 inlets, other quantities of inlets may be used as well.

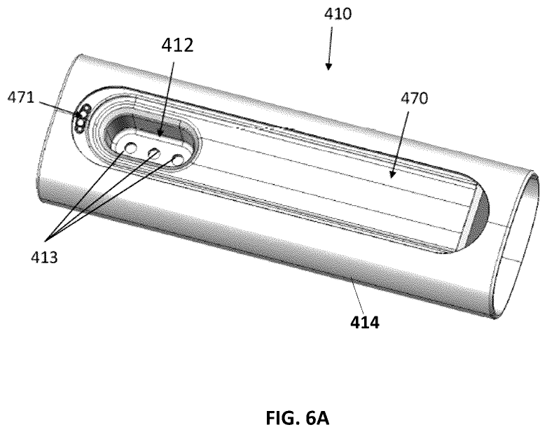

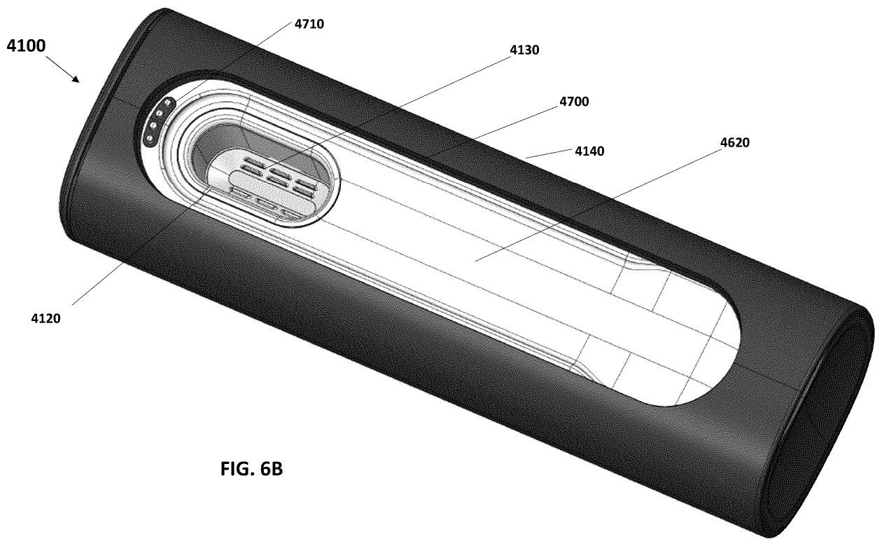

FIG. 6A illustrates a portion of the vaporizer device 410, consistent with implementations of the current subject matter. The oven chamber 412 includes one or more (e.g., a plurality, one, two, three, four, five, or more) oven chamber inlets 413. The oven chamber inlets 413 allow for the air, such as the heated air heated by the convection heating unit (described below) to enter the oven chamber 412 via the oven chamber inlets 413. In the oven chamber 412, the heated air may rapidly heat the vaporizable material to assist in vaporizing the vaporizable material. The recess 470 may include one or more coupling features, such as magnets. The magnets may be used to, for example, magnetically couple the cover 480 to the vaporizer body 414. Other coupling features are also contemplated to allow the cover to be easily attached and/or detached to allow access to at least the oven chamber 412. In some embodiments, the recess 470 may include one or more pins 471 (e.g., contacts) which may carry power and/or control signals. For example, the pins may couple to corresponding pins in a vaporizer cover to provide power to a concentrate oven located at the vaporizer cover, detect the placement of the vaporizer cover door in the recess, and/or detect a switch 481 on or off of the vaporizer and/or mode change of the vaporizer.

FIG. 6B illustrates a portion of the vaporizer device 4100, consistent with implementations of the current subject matter. The oven chamber 4120 includes one or more (e.g., a plurality, one, two, three, four, five, or more) oven chamber inlets 4130. The oven chamber inlets 4130 allow for the air, such as the heated air heated by the convection heating unit (described below) to enter the oven chamber 4120 via the oven chamber inlets 4130. In the oven chamber 4120, the heated air may rapidly heat the vaporizable material to assist in vaporizing the vaporizable material. The recess 4700 includes one or more coupling features, such as magnets. The magnets may be used to, for example, magnetically couple the cover 4800 to the vaporizer body 4140. For example, the magnets may couple to a corresponding set of magnets or metal at the cover. Alternatively, the magnets may be located at the cover, in which case the vaporizer body would include magnets or some other metal material. Other coupling features are also contemplated to allow the cover 4800 to be easily attached and/or detached to allow access to at least the oven chamber 412. In some embodiments, the recess 4700 may include one or more pins 4710 (e.g., contacts) which may carry power and/or control signals. For example, the pins may couple to corresponding pins in a vaporizer cover to provide power to a concentrate oven located at the vaporizer cover, detect the placement of the vaporizer cover door in the recess, and/or detect a switch on or off of the vaporizer and/or mode change of the vaporizer.

FIG. 6B also depicts a screen 4131A inserted into the base of the oven chamber 4120. The screen may include one or more inlets 4131B as well. The screen may be used to keep the oven chamber's base clean, when compared to not using the screen.

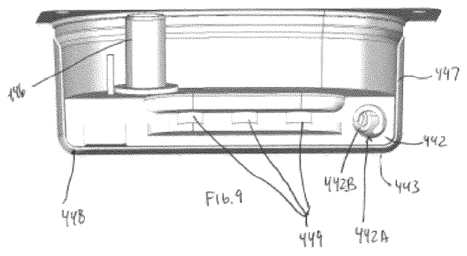

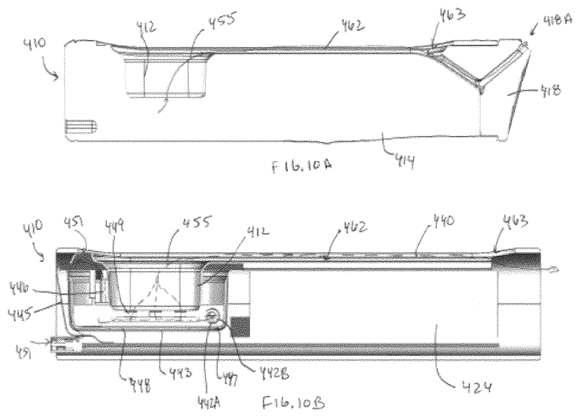

FIGS. 7A and 7B illustrate exploded views of the vaporizer device 410, consistent with implementations of the current subject matter. FIGS. 8A and 8B illustrate partial assembly views of the vaporizer device 410, consistent with implementations of the current subject matter, in which certain components were removed for case of view. As noted, one or more components for vaporizer 410 (at FIGS. 7A and 7B) may be the same (or similar) as or may be interchanged with one or more components of the vaporizer device 10, 310, and 4100 consistent with implementations of the current subject matter.

Referring to FIGS. 7A-7B and FIGS. 8A-8B, the vaporizer device 410 includes an inner cover 440 including the oven chamber 412 and the cooling pathway 462, a ceramic cover 441, a convection heater 442 (e.g., the convection heating unit), an inner chamber 447, an outer chamber 443, a heating element 444, a heating circuit 445, a tube 446, and a battery 424.

As described herein, the vaporizer device 410 includes a hybrid heating system. The hybrid heating system include the convection heating unit and a conduction heating unit. The hybrid heating system may thus rapidly heat the vaporizable material to provide on-demand or near on-demand heating of the vaporizable material and generation of aerosol, thereby improving the user experience. For example, the heating element 444 may be wrapped around and/or contact the oven chamber 412 (e.g., an external wall of the oven chamber) to heat the oven chamber 412, such as via conduction, as part of the conduction heating unit. The heated oven chamber 412 may contact the vaporizable material positioned within the oven chamber 412 to heat the vaporizable material. Additionally, and/or alternatively, air entering the vaporizer device 410 may pass through the convection heating unit 442 to heat the air prior to the air entering the oven chamber 412, where the heated air heats and/or vaporizes the vaporizable material. Thus, the vaporizable material may be heated via conduction and/or convection for rapid heating.

FIG. 9 illustrates a partial cross-sectional view of a portion of the vaporizer device 410, consistent with implementations of the current subject matter. As shown in FIG. 9, the inner chamber 447 may be positioned within the outer chamber 443. The inner chamber 447 may be separated from the outer chamber 443 by an air gap 448. The air gap 448 may insulate the inner chamber 447 from other components external relative to the outer chamber 443. For example, the inner chamber 447 and the outer chamber 443 may form an insulation unit. The air gap 448 may be sealed between the inner chamber 447 and the outer chamber 443 to limit or prevent heat from the convection heater 442 from impacting components external to the outer chamber 443. The inner chamber 447 and the outer chamber 443 may be made of stainless steel or another material to insulate the interior of the inner chamber 447. The convection heater 442 may include a heating channel 442A and a heating element 442B. The heating channel 442A may extend about a perimeter of the convection heater 442. The heating element 442B may be a spring coil heater (though other heaters are contemplated) that is positioned within the heating channel 442A. Ambient air may pass into the heating channel 442A, such as via the tube 446, to be heated by the heating element 442B before passing through the convection heater outlets 449 (e.g., one or more, such as one, two, three, four, five, or more outlets). The heated air exiting the convection heater 442 through the outlets 449 may pass into the oven chamber 412 to heat the vaporizable material.

FIGS. 10A and 10B illustrate cross-sectional views of the vaporizer device 410 and example airflow paths 455, consistent with implementations of the current subject matter. Ambient air may pass into the vaporizer device 410 via one or more inlets, such as inlets 451. As noted, ambient air may pass along the airflow path 455 into the heating channel 442A of the convection heater 442, such as via the tube 446, to be heated by the heating element 442B before passing through the convection heater outlets 449. The heated air exiting the convection heater 442 along the airflow path 455 through the outlets 449 and into the inlets 413 may pass into the oven chamber 412 to heat the vaporizable material. The heating element 444 may additionally and/or alternatively heat the oven chamber 412 via conduction, which in turn heats the vaporizable material via conduction. Accordingly, the hybrid heating system may rapidly heat the vaporizable material via convection and/or conduction. The vaporizable material may be rapidly heated via convection and/or conduction to generate an aerosol. The aerosol continues along the airflow path 455 along the cooling pathway 462 to the outlet 463, where the aerosol travels along the airflow path 455 to the outlet 418A of the mouthpiece 418, where the aerosol is rapidly delivered to the user.

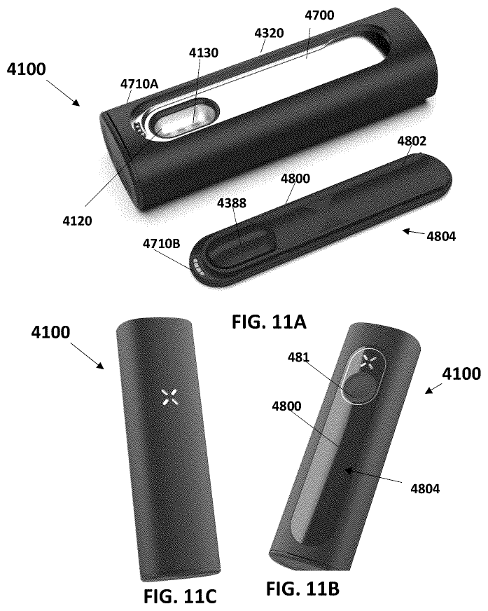

FIGS. 11A-11C depict another example of the vaporizer device 4100, without the mouthpiece inserted into the vaporizer body. FIG. 11B depicts the vaporizer device 4100 of FIG. 11A with the cover 4800 coupled to the vaporizer device 4100. FIG. 11C depicts the opposite side of the vaporizer device depicted at FIG. 11B. In the view shown at FIG. 11A, the vaporizer device 4100 is depicted with the cover 4800 removed (or detached) from the outer shell 4320 of the vaporizer device. When the cover 4800 is removed, the oven chamber 4120 is exposed. The cover 4800 may be removably coupled to the recess 4700 using for example magnets (which attract or couple to a metal or another magnet located at the cover, such that when the cover is placed into the recess 4700, magnetic attraction keeps the cover within the recess. The magnets may be located at other locations as well. Other coupling features are also contemplated to allow the cover to be easily attached and/or detached to allow access to at least the oven chamber. In some embodiments, the recess 4700 may include one or more pins 4710 (e.g., contacts) which may carry power and/or control signals. For example, the pins may couple to corresponding pins 4710B in a vaporizer cover to provide power to a concentrate oven located at the vaporizer cover, detect the placement of the vaporizer cover door in the recess, and/or detect a switch on or off of the vaporizer and/or mode change of the vaporizer.

The cover 4800 may be implemented in the same of similar manner noted above with respect to cover 380. The cover 4800 includes a first surface 4802, a second surface 4804 (which is distal to the first surface 4802), and a projection 4388. The first surface 4802 is configured to face towards an interior of the vaporizer body, such that the first surface 4802 faces the recess 4700, while the projection aligns with (or inserts into, covers, etc.) the opening of the oven chamber 4120. The projection 4388 may be a solid material, such that the projection 4386 compresses any vaporizable material within the oven chamber. This allows for improved, consistent, and/or even heating of the vaporizable material within the oven chamber. Additionally, and/or alternatively, the projection 4388 may include one or more tortuous features that create turbulent airflow of the generated aerosol passing from the oven chamber to the cooling pathway to cool the generated aerosol. Additionally, and/or alternatively, the oven chamber may include one or more (e.g., a plurality, one, two, three, four, five, or more) oven chamber inlets 4130. The oven chamber inlets 4130 allow for the air, such as the heated air heated by the convection heating unit (described below) to enter the oven chamber 4120 via the oven chamber inlets. In the oven chamber, the heated air may rapidly heat the vaporizable material to assist in vaporizing the vaporizable material.

In some embodiments, the cover (also referred to herein as the concentrate cover) of the vaporizer device may comprise a projection that extends into the oven chamber and compresses vaporizable material located in the oven chamber.

In some embodiments, the cover (also referred to herein as the concentrate cover) of the vaporizer device may comprise a concentrate oven (also referred to as a concentrate container) that is (1) sized to fit into the oven chamber and that is (2) configured to heat vaporizable material located in the concentrate oven. In some embodiments, the concentrate container is heated using a heater dedicated to the concentrate container. For example, when the concentrate cover 4800 is inserted in to the recess 4700, the pins 4710B of the concentrate cover and the pins 4710A of the recess make contact. This contact may be automatically detected. In response to this detection, the vaporizer body may provide power to the conduction heater (see, e.g., conduction heater 1370C) in contact with the concentrate oven 1202 to heat any concentrate in the concentrate oven. Alternatively, or additionally, the vaporizer oven may, during the heating of the concentrate oven 1202, stop providing power to heat the oven chamber 4120. Although the previous example refers to powering the concentrate oven based on the detection of the concentrate oven cover's pins 4710B, the powering of the concentrate oven may be based on other events, such as a button 481 toggle to change modes to concentrate oven mode rather than oven chamber mode. Although the previous example refers to heating the concentrate oven using only the heat from the concentrate oven, the concentrate oven may be heated using at least some of the heat produced by the oven chamber.

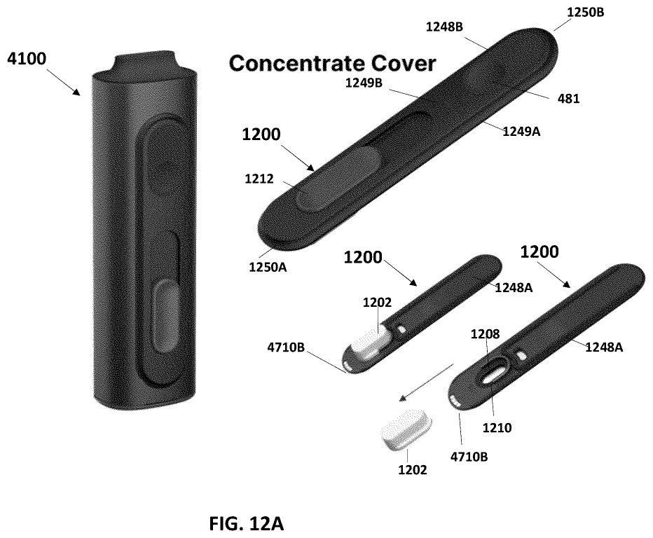

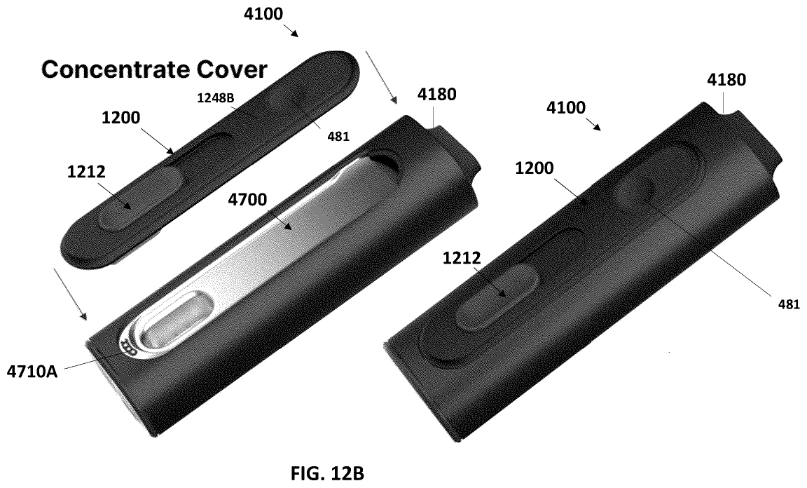

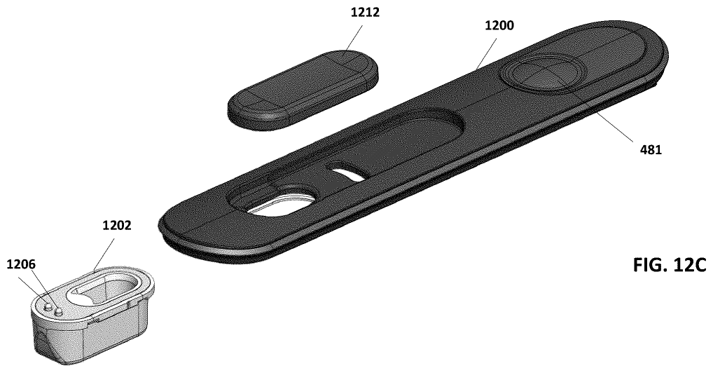

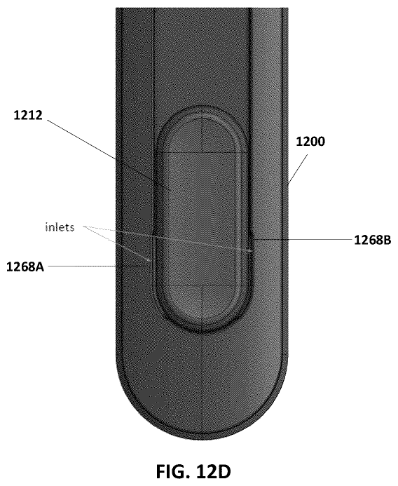

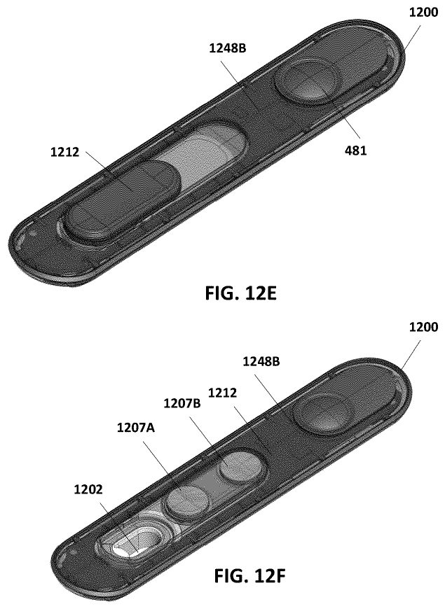

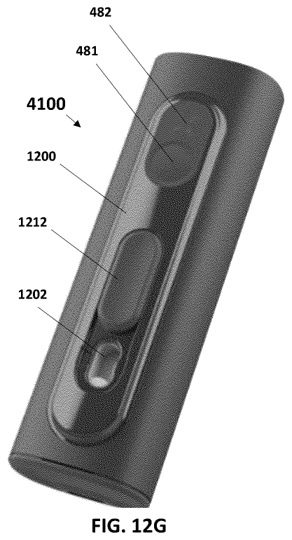

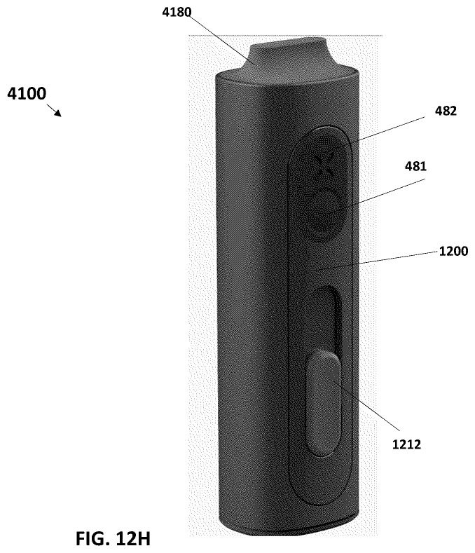

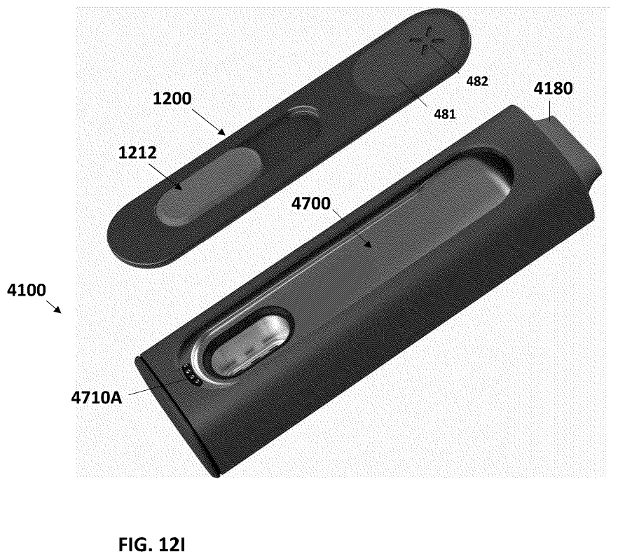

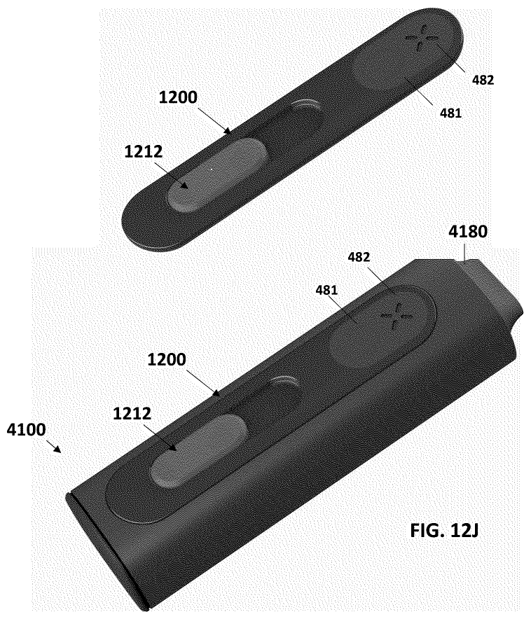

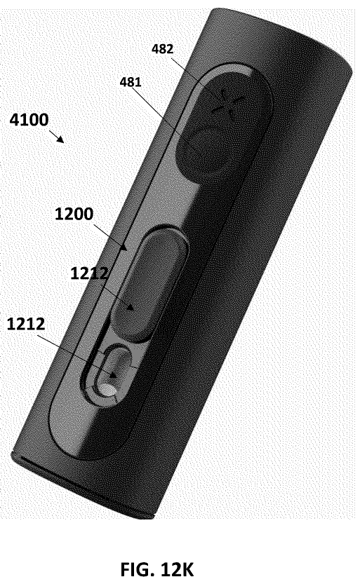

FIGS. 12A-12G depict examples of the vaporizer and cover 1200 including a concentrate container 1202. In the examples of FIGS. 12A-12G, the cover 1200 (also referred to as a “slider”) may include one or more magnet which physically couples the cover 1200 to the vaporizer body, as noted above with respect to FIGS. 11A-C, for example. In some embodiments, there may be one or more pins or contacts at 4710B that couple to corresponding pins or contacts located in the recess 4700 (e.g., pins or contacts 4710A).

In some embodiments, the concentrate container (also referred to as a concentrate oven) 1202 is sized to fit into the oven chamber 4120 (see, e.g., FIG. 11A).

In some embodiments, the concentrate container 1202 includes its own source of conduction heat, such as a coil or wire based heater in contact with or wrapped around a portion of the heater. Alternatively, or additionally, concentrate container 1202 uses the heat provided by the oven chamber 4120 to heat the vaporizable material in the concentrate container 1202. Alternatively, or additionally, the air flowing into the concentrate container may be pre-heated using the convection heater of the vaporizer device. In operation, a user of the vaporizer device may insert a concentrate or other vaporizable material into the concentrate container 1202 (such that when the cover 1200 is coupled to the vaporizer body 4100), and the concentrate container is then heated using a conduction heater dedicated to the concentrate container, the heat provided by the oven chamber, and/or a combination thereof. In a sense, the concentrate container serves as a concentrate oven for the vaporizable material in the concentrate container.

In the example of FIG. 12A, the concentrate cover 1200 includes a first surface 1248A and a second surface 1248B (which is distal to the first surface 1248A). The concentrate cover 1200 includes parallel long side, namely a first long side 1249A and an opposite second long side 1249B, and the cover further includes a first short side 1250A and an opposite second short side 1250B. The first surface 1248A is configured to face towards an interior of the vaporizer body, such that the first surface faces the recess of the vaporizer and aligns with and inserts (at least in part) into the opening of the oven chamber 4120. FIG. 12B illustrates a sequence of placing the concentrate cover 1200 into the recess 4700 of the vaporizer body.

Referring again to FIG. 12A, the concentrate oven 1202 may be fixedly attached to the cover 1200. Alternatively, or additionally, the concentrate oven 1202 may be removably attached. For example, the concentrate oven may slide into and become fixedly attached (e.g., a snap fit or other coupling) into a ridge structure 1208 that surrounds at least in part the opening 1210 of the cover.

The concentrate oven 1202 may include a concentrate oven cover 1212. The concentrate oven cover may be opened to add additional concentrate or closed as shown at FIG. 12A (e.g., before use of the vaporizer device). In the example of FIG. 12A, the concentrate chamber cover slides open and closed to expose the concentrate oven. The concentrate cover 1200 may also include a button 481, which as noted may be used to remove the concentrate cover from the recess 4700, power the vaporizer on or off, change vaporizer modes, and/or the like. FIGS. 12E and F depicts the concentrate oven cover opened (FIG. 12E) and slidably closed (FIG. 12F).

Referring to FIG. 12C, the concentrate oven 1202 may, as noted, above have its own source of conduction heating separate from the conduction heater of the oven chamber 4120 of the vaporizer body, for example. In the example depicted at FIG. 12C, the concentrate oven 1202 includes one or more electrical contacts 1206. These electrical contacts are used to provide power to the conduction heater located in the concentrate oven 1202. In operation, when the concentrate oven 1202 is inserted into the cover, the electrical contacts 1206 make contact with corresponding contacts (e.g., contacts or pins 4710B) in the concentrate cover 1200, which couple to a battery of the vaporizer body.

FIG. 12D depicts that the concentrate cover 1200 (and/or the concentrate oven cover 1212) may include one or more inlets, such as an inlet 1268A and an inlet 1268B. The inlets allow an air flow into at least the concentrate oven 1202.

FIGS. 12E-12F depict another example of the concentrate cover 1200, where FIG. 12E depicts the concentrate oven cover 1212 open, while FIG. 12F shows a view with the concentrate oven cover 1212 removed from the assembly shown. In the example of FIG. 12E, the concentrate oven cover 1212 may slide to expose the concentrate over 1202, and the cover 1200 may be removably attached to the vaporizer body using one or more magnets (or material which may be attracted to a magnet), such as the magnets 1207A-B. These magnets may couple to corresponding magnets (or magnetic material such as steel) located at the vaporizer body or recess.

FIG. 12G depicts the vaporizer device 4100 with the concentrate oven cover 1212 open to expose the oven chamber 1202. FIG. 12G also depicts a button 481. The button may be manipulated to couple or decouple the cover 1200 from the vaporizer body 4100. Alternatively, or additionally, the button may switch the power on or off at the vaporizer device 4100 (or the cover 4800). Alternatively, or additionally, the button may be used to change the operating modes of the vaporize device 4100. Alternatively, or additionally, the vaporizer may include a user interface (e.g., one or more LEDs 482), which can be used to provide indications to a user (e.g., an indication of power being on or off, an indication of a current mode of the vaporizer, an indication of a mode change, an indication of a heater temperature, and/or the like). FIG. 12H depicts the vaporizer device 4100 with the concentrate oven cover 1212, but unlike FIG. 12G the cover 1200 is flush with the vaporizer body and the concentrate oven cover (or slider) 1212 is closed. FIG. 12I depicts the vaporizer device 4100 with the concentrate oven cover 1212, but unlike FIG. 12B the cover 1200 is flush with the vaporizer body and the LEDs 482 are disposed on the cover 1200. FIG. 12J depicts the vaporizer device 4100 with the concentrate oven cover 1212 with the slider 1212 closed. FIG. 12K depicts the vaporizer device 4100 with the concentrate oven cover 1212 open to expose the concentrate container (or oven).

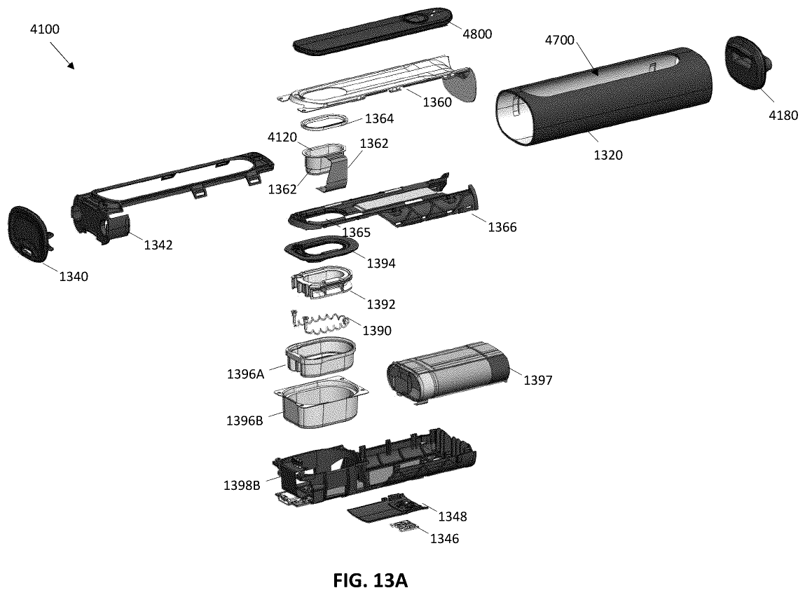

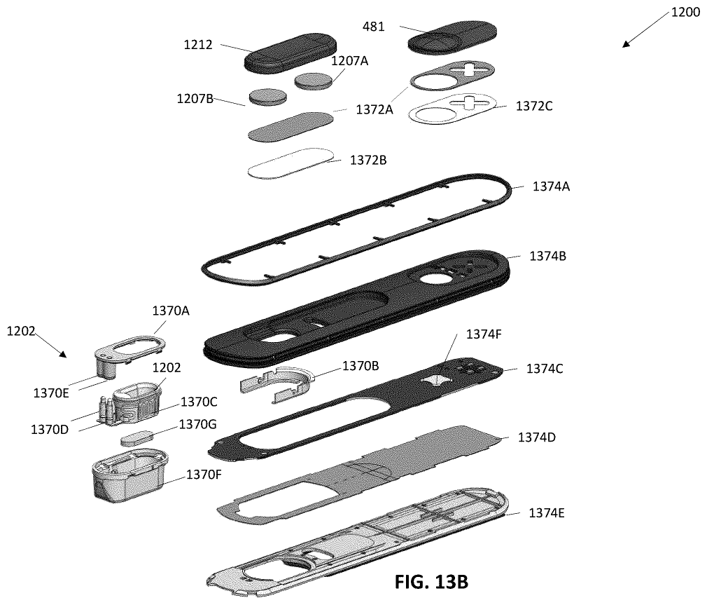

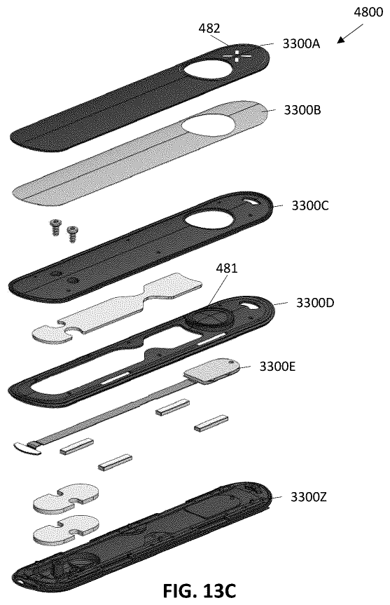

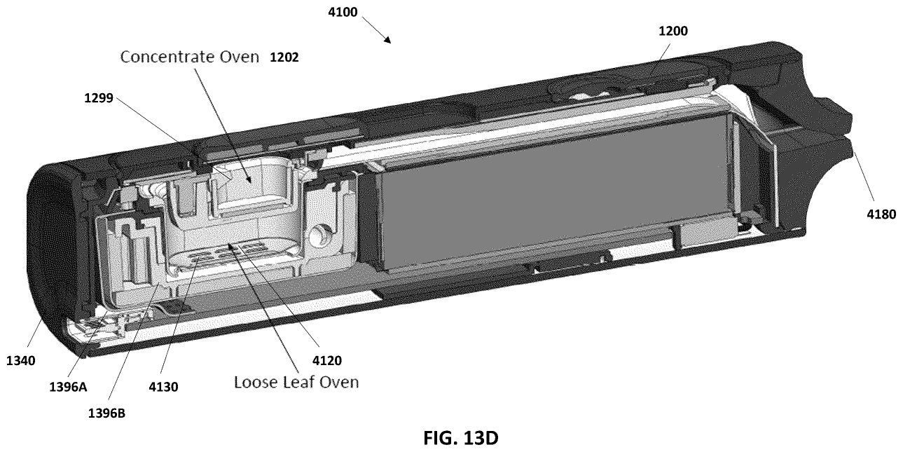

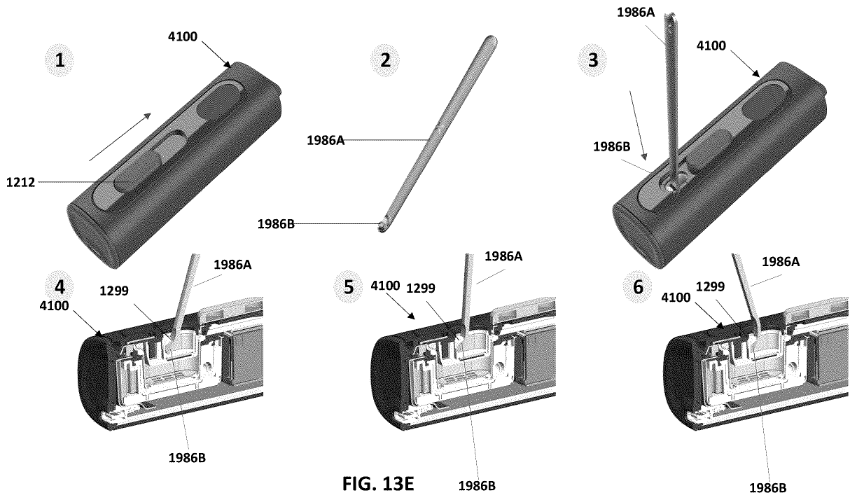

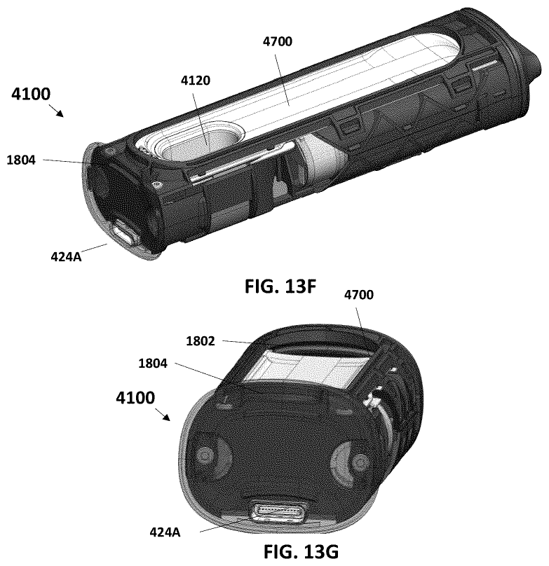

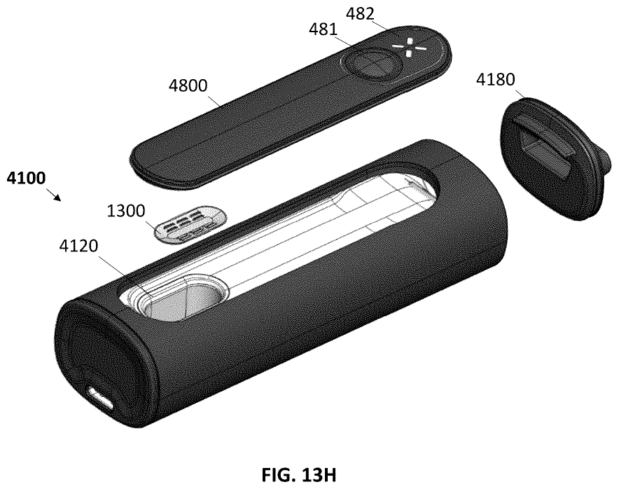

FIG. 13A-13G depict various views of the vaporizer device. FIGS. 13A-13B depict exploded views; FIG. 13C depicts an exploded view of the cover of the vaporizer device; FIG. 13D depicts a cross-sectional view of the vaporizer device; FIG. 13E depicts the vaporizer device while being loaded with concentrate material; and FIGS. 13F-13H depict various perspective views of the vaporizer device.

FIG. 13A depicts an exploded view of the vaporizer device 4100. Referring to FIG. 13A, the vaporizer device includes a mouthpiece 4180 and an outer shell 1320 with the recess 4700. The concentrate cover couples to an oven skeleton structure 1360. The oven chamber 4120 is coupled to a conduction heater's heating element 1362 that wraps (at least in part) around a portion of the oven chamber 4120. Alternatively, or additionally, the heating element may be located within the interior of the oven chamber and/or embedded at least in part in the oven chamber.

A heat break 1364 may be fitted between the top surface of the oven chamber and the bottom surface of the oven skeleton structure 1360 to dissipate heat and/or reduce heat transfer from the oven chamber 4120 to the oven skeleton structure 1360 and the outer shell 1320. The oven chamber 4120 inserts into and is affixed to an upper skeleton structure 1366 via an opening 1365. A convection heater 1390 is affixed to a convection oven 1392 such that the convection heater wraps around at least a portion of the convection oven. The convection heater 1390 and the convection oven 1392 couple to the upper skeleton structure 1366. A convention block seal 1394 may be placed between the convection oven 1392 and the upper skeleton structure 1366 to provide at least a partial air seal. In the exploded view, the convection oven 1392 (along with convection heater 1390) pre-heats air flowing into the oven chamber 4120, which is heated by conduction heaters 1362.

FIG. 13A depicts a lower convection block 1396A and a convection can 1396B. The lower convection block 1396A and the convection can 1396B affix to the upper skeleton structure 1366 and a lower skeleton structure 1398B (all of which is enclosed by the outer shell 1320). The lower convection block 1396A and the convection can 1396B (which further couple to the oven skeleton structure 1360) contain the convection heater assembly components (e.g., the convection heater 1390, the convection oven 1392, the convention block seal 1394, the lower convection block 1396A, and the convection can 1396B) and the conduction heater components (e.g., the oven chamber 4120 and the conduction heater 1362) as well as the vaporizer battery 1397 used to power the vaporizer device.

FIG. 13A also depicts an end cap 1340 for the vaporizer device and an end cap skeleton 1342 configured to couple to the end cap 1340. FIG. 13A also depicts a light pipe 1346 providing light from one or more LEDs and a light pipe divider 1348 which separates light (e.g., into different colors).

FIG. 13B depicts an exploded view of the cover 1200 including the concentrate oven (also referred to as concentrate container) 1202. The concentrate oven 1202 includes a conduction heater 1370C (e.g., heater traces affixed to the side of the concentrate oven 1202) coupled to heater pins 1370D. In the example of FIG. 13B, the heater pins 1370 insert into pin holes 1370E a retainer ring 1370A. The concentrate oven 1202 and retainer ring 1370A may be inserted or encased by the lower heater housing 1370F. A heat spacer 1370G (e.g., heat shield or insulator) may be placed between the lower surface of the concentrate oven 1202 and a bottom surface of the lower heater housing 1370F. The conduction heater 1370C provides a conductive heat source to vaporizable material located within the concentrate oven 1202. This conduction heater 1370C is separate from the conductive heater 1362 of oven chamber 4120. Moreover, the conduction heater 1370C may be separately powered. In use for example, the vaporizer device may be in a concentrate mode where concentrate is placed into the concentrate oven 1202. In this concentrate mode, the conduction heater 1370C may be powered on to heat the concentrate, and the conductive heater 1362 of oven chamber 4120 may be powered off. If the vaporizer is in an oven chamber mode where vaporizable material is instead placed in the oven chamber 4120, the conductive heater 1362 of oven chamber 4120 may be powered one while the concentrate's conduction heater 1370C may be powered off. To illustrate further, the concentrate oven/container 1202 is heated using a heater dedicated to the concentrate container. For example, when the concentrate cover 4800 is inserted into the recess 4700, the pins 1370D of the concentrate cover and the pins 4710A of the recess make contact. As noted, this contact may be automatically detected such that the vaporizer body controls the flow of power to the conduction heater 1370C, which is in contact with the concentrate oven 1202 to heat any concentrate in the concentrate oven. Alternatively, or additionally, the vaporizer may stop providing power to the vaporizer chamber 4120. Although the previous example refers to powering the concentrate oven based on the detection of the concentrate oven cover's pins, the powering of the concentrate oven may be based on other events, such as a button 481 toggle to change modes to concentrate oven mode rather than oven chamber mode. Although the previous example refers to heating the concentrate oven using only the heat from the concentrate oven, the concentrate oven may be heated using at least some of the heat produced by the oven chamber.

FIG. 13B also shows the concentrate oven cover 1212 including magnets 1207A-B, adhesive 1372A, and a metal end cap 1272B, which are all coupled together to form and provide the concentrate cover door. The button 481 is also depicted including the adhesive 1372A and another end cap 1372C, which are all coupled together to form and provide the button 481. The concentrate cover door slidably attaches to the metal skeleton 1374B, and the button 481 also attaches to the metal skeleton. A retainer ring 1370B is affixed to a bottom surface of the metal skeleton. The retainer ring is configured to enable the concentrate oven 1212 to be inserted (or removed) if needed from the concentrate oven cover. The metal skeleton may be circumscribed with a seal 1374A. A bottom layer skeleton 1374E may couple to the metal skeleton, such that the formed enclosure includes an adhesive layer 1374D and an intermediate layer 1374C (e.g., to carry electrical signals and/or power) including a printed circuit board (PCB) and a switch 1374F (e.g., dome switch) to control powering the device on or off as well as changing modes of the vaporizer. The printed circuit board may include circuitry for detecting the presence of the cover which includes the concentrate oven, carrying power signals, carrying control signals, and/or the like.

FIG. 13C shows an exploded view of the cover 4800 which does not include the concentrate container (concentrate oven) 1202. Referring to FIG. 13C, the cover 4800 may be formed using an upper skeleton 3300A and a lower skeleton 3300Z. The cover may include intermediate components, such as an adhesive layer 3300B, a button structure 3300D having the button 481, a printed circuit board layer 3300E for detecting the activations of the button 481 (e.g., turning the vaporizer device on or off, changing modes, changing temperatures, and/or the like) and/or carrying power signals to the heater(s).

FIG. 13D depicts a cross section view of the vaporizer device 4100 including the concentrate oven 1202. FIG. 13D also shows an application ridge 1299 located on the concentrate oven 1202. FIG. 13E depicts the cleaning ridge in operation. In panel 1, the concentrate oven cover 1212 is opened to expose the concentrate oven 1202. Panel 2 depicts an applicator 1986A with a concentrate material 1986B. At panels 3-6, the applicator and the concentrate material are inserted into the concentrate oven. At panel 4, the applicator 1986A makes contact with the application ridge 1299, so the heat from concentrate oven also heats the applicator (and/or concentrate material) so the concentrate material flows concentrate oven and is vaporized. Alternatively, or additionally, the cleaning ridge can be used to clean the applicator of concentrate material. The application ridge is located at an upper portion opposite a base portion of the concentrate oven chamber. FIGS. 13F and 13G depict, as noted, perspective views of the vaporizer device 4100 and show a vaporizer inlet 1804 (where air can be sucked in) and the vaporizer inlet 1802 (which is the end the vaporizer's mouthpiece is coupled). FIG. 13H depicts concentrate cover 4800 (see, e.g., FIG. 13C) without a concentrate oven as well as screen 1300 being inserted into the oven chamber 4120.

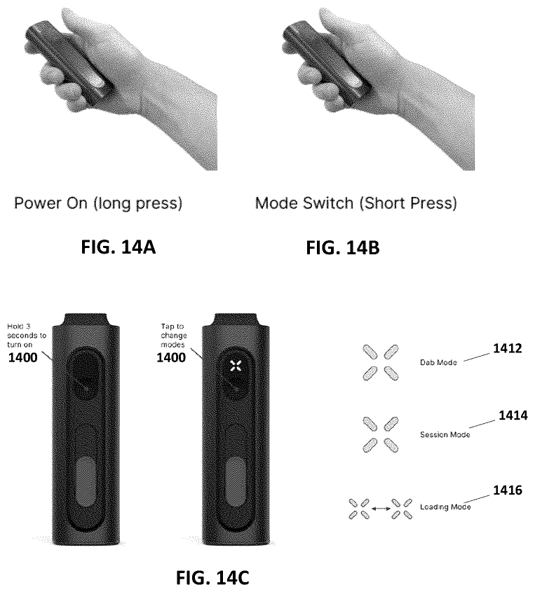

FIGS. 14A-C depicts the vaporizer device 4100 in operation. For example, a button 1400 may be depressed in a predetermined pattern to change the mode of the vaporizer device 4100. For example, button 1400 may be pressed for a predetermined amount of time above a threshold amount of time (e.g., 1 second, 2 seconds, 3 seconds, or other times), such that this long press powers the vaporizer device “on” as shown at FIG. 14A. In the example of FIG. 14B, the button 1400 may be pressed for less than the predetermined amount of time to step through or change the modes of the vaporizer. For example, a first short press (which is less than the predetermined time) may configured the vaporizer device for a first mode 1412 (e.g., a dab mode for concentrate material heating of the concentrate oven 1202), a second press may configure the vaporizer for a second mode 1414 (e.g., a flower mode for heating of flower or other vaporizable material in the oven chamber 4120), a third press may configure the vaporizer device for a third mode 1416 (e.g., a loading mode, a cleaning mode, and/or other modes). Each mode may correspond to a different heating temperature of the vaporizer device. Moreover, the change in mode may be signaled or indicated to the user via haptics or a predetermined indication on one or more LEDs 1420 of the vaporizer device.

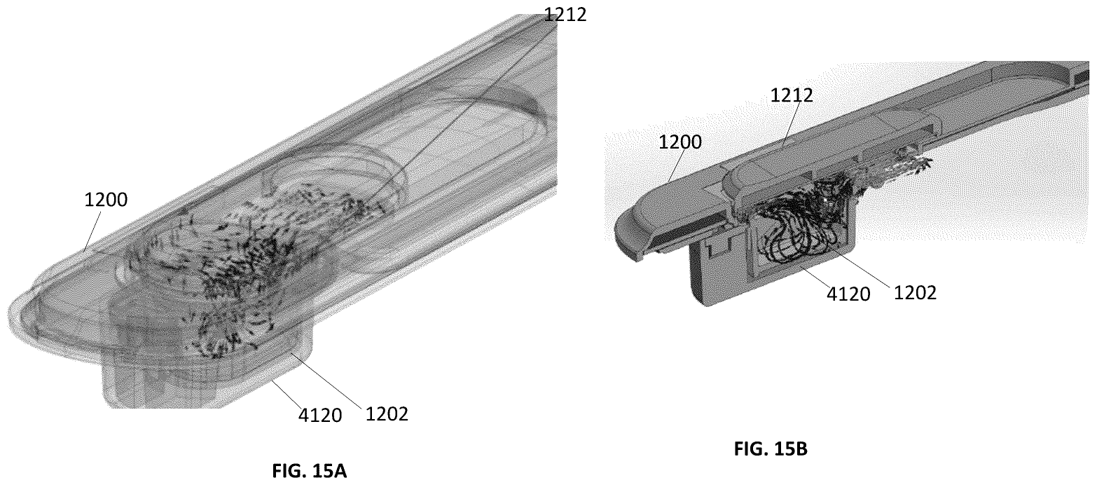

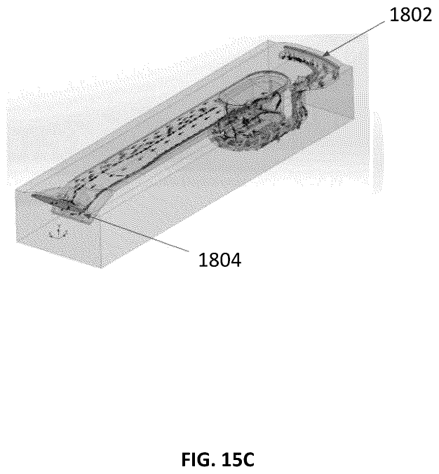

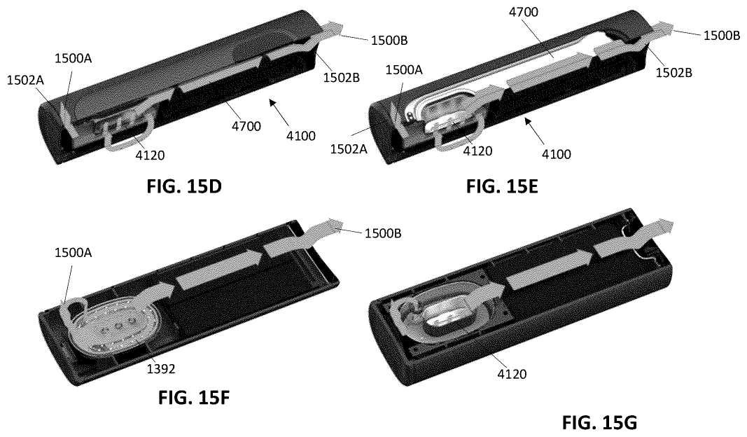

FIGS. 15A-C depicts examples of the airflow associated with the vaporizer device. At FIG. 15A, the concentrate oven 1202 is in the oven chamber 4120, so as a user takes a puff air is drawn into at least the inlets 1212. The airflow is depicted by the lines with arrows. FIG. 15B depicts a cutaway view of FIG. 15A. FIG. 15A and FIG. 15B illustrate example airflow paths of a concentrate oven for a vaporizer device, consistent with implementations of the current subject matter. As shown, the air flows along the top of the oven cup of the concentrate oven, and negative pressure pulls vapor into the air stream and across the airflow path to the mouthpiece. The concentrate oven door 1200 (also referred to as a slider) may include one or more inlets, such as inlet 1212 for the airflow path or at other positions around the slider.

FIG. 15C depicts an overall flow from the vaporizer inlet 1804 by the mouthpiece and the vaporizer outlet 1802 (which is the end the vaporizer's mouthpiece is coupled). In this example, the vaporizer device is used for heating a vaporizable material stored in the reservoir (e.g., oven 4120) of the vaporizer device body. As shown, the air flows through the inlet, around the convection coil, underneath and up into the oven, through the oven, and across the air pathway to the mouthpiece. The airflow path bends down towards the mouthpiece to at least improve case of cleaning and assist with pivoting the cover. Further, the mouthpiece positioning completes a seal with the cover such that the airflow path is sealed within the vaporizer device and leakage is prevented or limited.

FIG. 15D-G depict additional, simplified view of the airflow 1500A-B. At FIG. 15D, the airflow 1500A enters at least one inlet 1502A and travels to the oven chamber 4120. Specifically, the air is pre-heated by the convection oven/heater 1390/1392 before entering the oven chamber and travels up towards the recess and out as airflow 1500B via the inlet 1502B (where a mouthpiece is coupled).

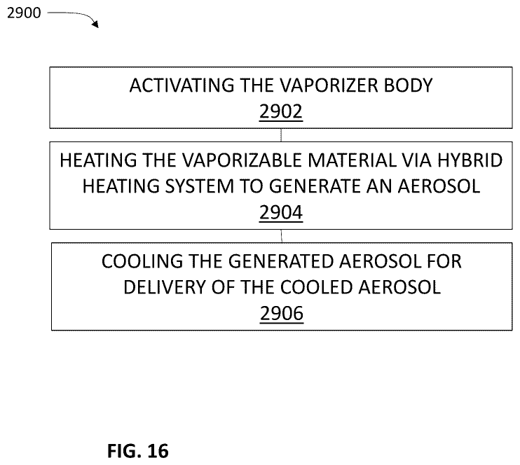

FIG. 16 is a chart shown an example method 2900 for generating an aerosol consistent with implementations of the current subject matter. While the method 2900 is described with respect to the vaporizer device 410, the method 2900 may apply to the vaporizer device 10, 310, and 4100.

At 2902, the vaporizer device 410, such as the vaporizer body 414, may be activated. For example, the vaporizer device 410 may be activated by drawing (e.g., inhaling) through the mouthpiece 418, receipt of contact with a button or another portion of the vaporizer device 410, such as the mouthpiece, receipt of a signal from a device in communication with the vaporizer device 410, and/or the like. In some implementations, the device 410 may detect a draw (e.g., using a pressure sensor, a capacitance sensor, flow sensors, a sensor configured to detect a change in temperature or power applied to the heating element, and/or the like) and may increase the power to a predetermined temperature preset. The power may be regulated by the controller of the vaporizer device 410. Consistent with implementations of the current subject matter, the vaporizer device 410 includes an oven chamber 412 configured to hold a vaporizable material. The vaporizer device 310 also includes a cover 480 having a projection 486 configured to extend into and enclose the oven chamber 412.

At 2904, the vaporizable material may be heated, such as by the hybrid heating system. For example, the convection heater 442 (e.g., the coil 442B) may activate (e.g., receive supplied power) to heat the air passing through the convection heater 442, which may then, via convection, heat the vaporizable material. Additionally and/or alternatively, the heating element 445 may activate (e.g., receive supplied power) to heat the oven chamber 412, and as a result, the vaporizable material in contact with the oven chamber 412, via conduction. Thus, activation of the hybrid heating system including the convection heater 442 and/or the heating element 445 may cause at least a portion of the vaporizable material stored within the vaporizer device 410 to be heated to generate an aerosol.

At 2906, the generated aerosol is cooled for delivery of the cooled aerosol to the user. For example, the generated aerosol may be passed along the cooling pathway 462 to further cool the generated aerosol before the aerosol is delivered through the mouthpiece 418 to the user.

In view of the above-described implementations of subject matter this application discloses the following list of examples, wherein one feature of an example in isolation or more than one feature of said example taken in combination and, optionally, in combination with one or more features of one or more further examples are further examples also falling within the disclosure of this application:
Example 1. A vaporizer device comprising: a mouthpiece; a hybrid heating system comprising a convection heater and a conduction heater; an oven chamber configured to contain a vaporizable material, wherein the vaporizable material is configured to be heated by convection, via air heated by the convection heater and by conduction, via contact with the oven chamber, wherein the oven chamber is configured to be heated by conduction using a heating element of the conduction heater, the heating element being in contact with the oven chamber; a vaporizer body comprising an outer shell which surrounds at least in part one or more internal components of the vaporizer body, the vaporizer body including a first end at which the mouthpiece is positioned and a second end opposite the first end, wherein the outer shell extends between the first end and the second end, the outer shell having two opposing long side portions and two opposing short side portions that define the outer shell, wherein the outer shell includes a recess exposing an interior of the vaporizer body, wherein the oven chamber is positioned in the vaporizer body and is exposed by the recess; and a vaporizer cover configured to be removable from the vaporizer body and configured to enclose at least the recess and the oven chamber.
Example 2. The vaporizer device of Example 1, wherein the recess is positioned along a length of the vaporizer body.
Example 3. The vaporizer device of any of Examples 1-2, wherein the length runs parallel to the two opposing long side portions of the vaporizer body.
Example 4. The vaporizer device of any of Examples 1-3, wherein long sides of the vaporizer cover are parallel to the two opposing long side portions of the outer shell.
Example 5. The vaporizer device of any of Examples 1-4, wherein the vaporizer cover includes an outer surface and an inner surface, wherein the outer surface is configured to face away from the vaporizer body, wherein the inner surface is opposite the outer surface and is configured to face towards an interior of the vaporizer body.
Example 6. The vaporizer device of any of Examples 1-5, wherein the inner surface includes a solid projection.
Example 7. The vaporizer device of any of Examples 1-6, wherein the solid projection extends from the inner surface into an interior volume of the oven chamber to enable compression of a vaporizable material located within the oven chamber.
Example 8. The vaporizer device of any of Examples 1-7, wherein the projection is shaped to correspond to a shape of the interior volume of the oven chamber.
Example 9. The vaporizer device of any of Examples 1-8, wherein the vaporizer cover includes a concentrate oven disposed on the inner surface of the vaporizer cover.
Example 10. The vaporizer device of any of Examples 1-9, wherein the concentrate oven extends from the inner surface of the vaporizer cover into the oven chamber.
Example 11. The vaporizer device of any of Examples 1-10, wherein the concentrate oven is removably attached to the inner surface of the vaporizer cover.
Example 12. The vaporizer device of any of Examples 1-11, wherein the concentrate oven includes a second conduction heater separate from the conduction heater used by the oven chamber.
Example 13. The vaporizer device of any of Examples 1-12, wherein the vaporizer cover includes a concentrate oven cover disposed on the outer surface of the vaporizer cover, wherein the concentrate oven cover is slidably attached to the vaporizer cover to expose the concentrate oven.
Example 14. The vaporizer device of any of Examples 1-13, wherein the concentrate oven is heated at least in part using the hybrid heating system of the vaporizer device.
Example 15. The vaporizer device of any of Examples 1-14, wherein the concentrate oven includes one or more inlets to enable air flow into the concentrate oven.
Example 16. The vaporizer device of any of Examples 1-15, wherein the vaporizer cover magnetically couples to the vaporizer body using at least one magnet.
Example 17. The vaporizer device of any of Examples 1-16, wherein the oven chamber comprises one or more oven chamber inlets to allow for air to enter the oven chamber via the one or more oven chamber inlets.
Example 18. The vaporizer device of any of Examples 1-17, wherein the air is pre-heated by the convection heater before entering the oven chamber via one or more inlets.
Example 19. The vaporizer device of any of Examples 1-18, wherein the mouthpiece comprises an outlet through which aerosol is delivered to a user of the mouthpiece.
Example 20. The vaporizer device of any of Examples 1-19, wherein the mouthpiece is tapered between two opposing long side portions of the mouthpiece.
Example 21. The vaporizer of any of Examples 1-20, wherein the vaporizer device comprises a cooling pathway formed between the oven chamber and the mouthpiece.
Example 22. The vaporizer of any of Examples 1-21, wherein cooling pathway includes one or more features to create a turbulent path for an airflow from the oven chamber to the mouthpiece.
Example 23. The vaporizer of any of Examples 1-22, wherein the heating element is wrapped around at least a portion of the oven chamber.
Example 24. A vaporizer device comprising: a mouthpiece; a hybrid heating system comprising a convection heater and a conduction heater; an oven chamber configured to contain a vaporizable material, wherein the vaporizable material is configured to be heated by convection, via air heated by the convection heater and by conduction, via contact with the oven chamber, wherein the oven chamber is configured to be heated by conduction using a heating element of the conduction heater, the heating element being in contact with the oven chamber; a vaporizer body comprising an outer shell which surrounds at least in part one or more internal components of the vaporizer body, the vaporizer body including a first end at which the mouthpiece is positioned and a second end opposite the first end, wherein the outer shell extends between the first end and the second end, the outer shell having two opposing long side portions and two opposing short side portions that define the outer shell, wherein the outer shell includes a recess exposing an interior of the vaporizer body, wherein the oven chamber is positioned in the vaporizer body and is exposed by the recess; and a vaporizer cover including an outer surface and an inner surface, wherein the outer surface is configured to face away from the vaporizer body, wherein the inner surface is opposite the outer surface and is configured to face towards an interior of the vaporizer body, and wherein the vaporizer cover includes a concentrate oven disposed on the inner surface of the vaporizer cover.
Example 25. The vaporizer device of Example 24, wherein the concentrate oven extends from the inner surface of the vaporizer cover into the oven chamber.
Example 26. The vaporizer device of any of Examples 24-25, wherein the concentrate oven is removably attached to the inner surface of the vaporizer cover.
Example 27. The vaporizer device of any of Examples 24-26, wherein the concentrate oven includes a second conduction heater separate from the conduction heater used by the oven chamber.
Example 28. The vaporizer device of any of Examples 24-27, wherein the vaporizer device is configured to provide power to the concentrate oven rather than the oven chamber.
Example 29. The vaporizer device of any of Examples 24-28, wherein in response to a mode change of the vaporizer or an automatic detection of the vaporizer cover including the concentrate oven being inserted into the recess, the vaporizer provides power to the concentrate oven.
Example 30. A vaporizer cover comprising: a button disposed on an outer surface of the vaporizer cover; a concentrate oven disposed on an inner surface of the vaporizer cover; and one or more pins disposed on the inner surface of the vaporizer cover, wherein the vaporizer cover includes the outer surface and the inner surface, wherein the outer surface is configured to face away from a vaporizer body when the vaporizer body is coupled to the vaporizer body and the inner surface is opposite the outer surface and is configured to face towards an interior volume of the vaporizer body.
Example 31. The vaporizer cover of Example 30, wherein the concentrate oven is configured to extend from the inner surface of the vaporizer cover into an oven chamber of a vaporizer device.
Example 32. The vaporizer cover of any of Examples 30-31, wherein the concentrate oven is removably attached to the inner surface of the vaporizer cover.
Example 33. The vaporizer cover of any of Examples 30-32, wherein the concentrate oven includes a second conduction heater configured to receive power via the one or more pins of the vaporizer cover.
Example 34. The vaporizer cover of any of Examples 30-33, wherein the vaporizer cover is shaped to fit in a recess of a vaporizer device.
Example 35. The vaporizer cover of any of Examples 30-34 further comprising: a user interface comprising one or more LEDs disposed on the outer surface.
Example 36. The vaporizer cover of any of Examples 30-35, wherein in response to a mode change of the vaporizer or an automatic detection of the vaporizer cover including the concentrate oven being inserted into a recess, the vaporizer cover receives power for the concentrate oven.
Example 37. The vaporizer cover of any of Examples 30-37, wherein one or more modes of the mode change include a first mode to power the concentrate oven and/or a second mode to power an oven chamber of a vaporizer device.

In some examples, the vaporizable material may include a plant material, such as a cannabis plant material. Additionally and/or alternatively, in some examples, the vaporizable material may include a viscous liquid such as, for example a cannabis oil and/or concentrate (e.g., wax, shatter, budder, butane hash oil, and the like). In some variations, the viscous liquid comprises between 0.3% and 100% cannabis oil extract. The viscous liquid may include a carrier for improving vapor formation, such as, for example, propylene glycol, glycerol, medium chain triglycerides (MCT) including lauric acid, capric acid, caprylic acid, caproic acid, etc., at between 0.01% and 25% (e.g., between 0. 1% and 22%, between 1% and 20%, between 1% and 15%, and/or the like). In some variations the vapor-forming carrier is 1,3-Propanediol. A cannabis liquid may include a cannabinoid or cannabinoids (natural and/or synthetic), and/or a terpene or terpenes derived from organic materials such as for example fruits and flowers. For example, any of the vaporizable materials described herein may include one or more (e.g., a mixture of) cannabinoid including one or more of: CBG (Cannabigerol), CBC (Cannabichromenc), CBL (Cannabicyclol), CBV (Cannabivarin), THCV (Tetrahydrocannabivarin), CBDV (Cannabidivarin), CBCV (Cannabichromevarin), CBGV (Cannabigerovarin), CBGM (Cannabigerol Monomethyl Ether), Tetrahydrocannabinol, Cannabidiol (CBD), Cannabinol (CBN), Tetrahydrocannabinolic Acid (THCA), Cannabidioloc Acid (CBDA), Tetrahydrocannabivarinic Acid (THCVA), one or more Endocannabinoids (e.g., anandamide, 2-Arachidonoylglycerol, 2-Arachidonyl glyceryl ether, N-Arachidonoyl dopamine, Virodhamine, Lysophosphatidylinositol), and/or a synthetic cannabinoids such as, for example, one or more of: JWH-018, JWH-073, CP-55940, Dimethylheptylpyran, HU-210, HU-331, SR144528, WIN 55,212-2, JWH-133, Levonantradol (Nantrodolum), and AM-2201. The oil vaporization material may include one or more terpene, such as, for example, Hemiterpenes, Monoterpenes (e.g., geraniol, terpincol, limonene, myrcene, linalool, pinene, Iridoids), Sesquiterpenes (e.g., humulene, farnesenes, farnesol), Diterpenes (e.g., cafestol, kahweol, cembrene and taxadiene), Sesterterpenes, (e.g., geranylfarnesol), Triterpenes (e.g., squalene), Sesquarterpenes (e.g, forrugicadiol and tetraprenylcurcumene), Tetraterpenes (lycopene, gamma-carotene, alpha- and beta-carotenes), Polyterpenes, and Norisoprenoids. For example, a liquid (e.g., oil and/or concentrate) vaporization material as described herein may include between 0.3-100% cannabinoids (e.g., 0.5-98%, 10-95%, 20-92%, 30-90%, 40-80%, 50-75%, 60-80%, etc.), 0-40% terpenes (e.g., 130%, 10-30%, 10-20%, etc.), and 0-25% carrier (e.g., medium chain triglycerides (MCT)).

In any of the liquid and/or semi-liquid vaporizable materials described herein (including in particular, the cannabinoid-based vaporizable materials), the viscosity may be within a predetermined range. The range may be between, at room temperature (23° C.) about 30 cP (centipoise) and 115 kcP (kilocentipoise), between 30cP and 200 kcP, although higher viscosities and/or lower viscosities may be implemented as well. For example, the viscosity may be between 40 cP and 113 kcP at room temperature. Outside of this range, the vaporizable material may fail in some instances to wick appropriately to form a vapor as described herein. In particular, it is typically desired that the liquid and/or semi-liquid may be made sufficiently thin to both permit transporting at a rate that is useful with the apparatuses described herein, while also limiting leaking (e.g., viscosities below that of ˜30 cP at room temperature might result in problems with leaking).

Although the disclosure, including the figures, described herein may described and/or exemplify these different variations separately, it should be understood that all or some, or components of them, may be combined.

Although various illustrative embodiments are described above, any of a number of changes may be made to various embodiments. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the claims.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. References to a structure or feature that is disposed “adjacent” another feature may have portions that overlap or underlie the adjacent feature.

Terminology used herein is for the purpose of describing particular embodiments only and is not intended to be limiting. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as, for example, “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings provided herein.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” “or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, are possible.

In the descriptions above and in the claims, phrases such as, for example, “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The implementations set forth in the foregoing description do not represent all implementations consistent with the subject matter described herein. Instead, they are merely some examples consistent with aspects related to the described subject matter. Although a few variations have been described in detail herein, other modifications or additions are possible. In particular, further features and/or variations can be provided in addition to those set forth herein. For example, the implementations described above can be directed to various combinations and sub-combinations of the disclosed features and/or combinations and sub-combinations of one or more features further to those disclosed herein. In addition, the logic flows depicted in the accompanying figures and/or described herein do not necessarily require the particular order shown, or sequential order, to achieve desirable results. The scope of the following claims may include other implementations or embodiments.

1. A vaporizer device comprising:
a mouthpiece;
a hybrid heating system comprising a convection heater and a conduction heater;
an oven chamber configured to contain a vaporizable material, wherein the vaporizable material is configured to be heated by convection, via air heated by the convection heater and by conduction, via contact with the oven chamber, wherein the oven chamber is configured to be heated by conduction using a heating element of the conduction heater, the heating element being in contact with the oven chamber;
a vaporizer body comprising an outer shell which surrounds at least in part one or more internal components of the vaporizer body, the vaporizer body including a first end at which the mouthpiece is positioned and a second end opposite the first end,
wherein the outer shell extends between the first end and the second end, the outer shell having two opposing long side portions and two opposing short side portions that define the outer shell,
wherein the outer shell includes a recess exposing an interior of the vaporizer body,
wherein the oven chamber is positioned in the vaporizer body and is exposed by the recess; and
a vaporizer cover configured to be removable from the vaporizer body and configured to enclose at least the recess and the oven chamber.

2. The vaporizer device of claim 1, wherein the recess is positioned along a length of the vaporizer body.

3. The vaporizer device of claim 2, wherein the length runs parallel to the two opposing long side portions of the vaporizer body.

4. The vaporizer device of claim 1, wherein long sides of the vaporizer cover are parallel to the two opposing long side portions of the outer shell.

5. The vaporizer device of claim 1, wherein the vaporizer cover includes an outer surface and an inner surface, wherein the outer surface is configured to face away from the vaporizer body, wherein the inner surface is opposite the outer surface and is configured to face towards an interior of the vaporizer body.

6. The vaporizer device of claim 5, wherein the inner surface includes a solid projection.

7. The vaporizer device of claim 6, wherein the solid projection extends from the inner surface into an interior volume of the oven chamber to enable compression of a vaporizable material located within the oven chamber.

8. The vaporizer device of claim 7, wherein the projection is shaped to correspond to a shape of the interior volume of the oven chamber.

9. The vaporizer device of claim 5, wherein the vaporizer cover includes a concentrate oven disposed on the inner surface of the vaporizer cover.

10. The vaporizer device of claim 9, wherein the concentrate oven extends from the inner surface of the vaporizer cover into the oven chamber.

11. (canceled)

12. The vaporizer device of claim 9, wherein the concentrate oven includes a second conduction heater separate from the conduction heater used by the oven chamber.

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. A vaporizer device comprising:
a mouthpiece;
a hybrid heating system comprising a convection heater and a conduction heater;
an oven chamber configured to contain a vaporizable material, wherein the vaporizable material is configured to be heated by convection, via air heated by the convection heater and by conduction, via contact with the oven chamber, wherein the oven chamber is configured to be heated by conduction using a heating element of the conduction heater, the heating element being in contact with the oven chamber;
a vaporizer body comprising an outer shell which surrounds at least in part one or more internal components of the vaporizer body, the vaporizer body including a first end at which the mouthpiece is positioned and a second end opposite the first end,
wherein the outer shell extends between the first end and the second end, the outer shell having two opposing long side portions and two opposing short side portions that define the outer shell,
wherein the outer shell includes a recess exposing an interior of the vaporizer body,
wherein the oven chamber is positioned in the vaporizer body and is exposed by the recess; and
a vaporizer cover including an outer surface and an inner surface, wherein the outer surface is configured to face away from the vaporizer body, wherein the inner surface is opposite the outer surface and is configured to face towards an interior of the vaporizer body, and
wherein the vaporizer cover includes a concentrate oven disposed on the inner surface of the vaporizer cover.

25. The vaporizer device of claim 24, wherein the concentrate oven extends from the inner surface of the vaporizer cover into the oven chamber.

26. The vaporizer device of claim 24, wherein the concentrate oven is removably attached to the inner surface of the vaporizer cover.

27. The vaporizer device of claim 24, wherein the concentrate oven includes a second conduction heater separate from the conduction heater used by the oven chamber.

28. The vaporizer device of claim 24, wherein the vaporizer device is configured to provide power to the concentrate oven rather than the oven chamber.

29. The vaporizer device of claim 28, wherein in response to a mode change of the vaporizer or an automatic detection of the vaporizer cover including the concentrate oven being inserted into the recess, the vaporizer provides power to the concentrate oven.

30. A vaporizer cover comprising:
a button disposed on an outer surface of the vaporizer cover;
a concentrate oven disposed on an inner surface of the vaporizer cover; and
one or more pins disposed on the inner surface of the vaporizer cover,
wherein the vaporizer cover includes the outer surface and the inner surface,
wherein the outer surface is configured to face away from a vaporizer body when the vaporizer body is coupled to the vaporizer body and the inner surface is opposite the outer surface and is configured to face towards an interior volume of the vaporizer body.

31. The vaporizer cover of claim 30, wherein the concentrate oven is configured to extend from the inner surface of the vaporizer cover into an oven chamber of a vaporizer device.

32. (canceled)

33. The vaporizer cover of claim 30, wherein the concentrate oven includes a second conduction heater configured to receive power via the one or more pins of the vaporizer cover.

34. (canceled)

35. (canceled)

36. (canceled)

37. (canceled)
